(19) United States
(12) Patent Application Publication
(10) Pub. No.: US 2012/0047602 A1
(43) Pub. Date: Feb. 23, 2012

(54) RNA-Mediated Induction of Gene Expression in Plants

(75) Inventors: Vinitha Joyce Cardoza, Morrisville, NC (US); Peifeng Ren, Cary, NC (US); Lawrence Winfield Talton, Cary, NC (US)

(73) Assignee: BASF Plant Science Company GmbH, Ludwigshafen (DE)

(21) Appl. No.: 13/264,886

(22) PCT Filed: Apr. 16, 2010
(86) PCT No.: PCT/EP2010/055009

§ 371 Date: Oct. 17, 2011

Related U.S. Application Data
(60) Provisional application No. 61/171,097, filed on Apr. 21, 2009.

(51) Int. Cl. A01H 5/00; C12N 1/21; C12N 15/113; C12N 5/10; C12N 15/82; C12Q 1/68

(52) U.S. Cl. 800/278; 435/6.12; 536/24.1; 435/320.1; 800/298; 435/419; 435/252.2; 435/468

(57) ABSTRACT

The present invention is in the field of plant genetics and provides methods for increasing gene expression of a target gene in a plant or part thereof. In addition the invention relates to methods for modifying the specificity of plant specific promoters and for engineering small non-coding activating RNA (sncaRNA) in order to increase expression of a target gene in a plant or part thereof. The present invention also provides methods for the identification of sncaRNA, and its primary transcripts in a plant capable of increasing gene expression in a plant or part thereof.

Many factors affect gene expression in plants and other eukaryotic organisms. Recently, small RNAs of 21 to 26 nucleotides have been found to be important repressors of eukaryotic gene expression. The known small regulatory RNAs fall into two basic classes: short interfering RNAs (siRNAs) and microRNAs.

MicroRNAs have emerged as evolutionarily conserved, RNA-based regulators of gene expression in animals and plants. MicroRNAs (approx. 18 to 25 nt) arise from larger precursors, pre-miRNAs, with a stem loop structure that are transcribed from non-protein-coding genes.

Plant microRNAs known so far repress expression of a high number of genes which function in developmental processes, indicating that microRNA-based regulation is integral to pathways governing growth and development. Gene expression-repressing plant microRNAs usually contain near-perfect complementarity with target sites, which occur most commonly in protein-coding regions of mRNAs (Llave C et al. (2002) Science 297, 2053-2056; Rhoades M W et al. (2002) Cell 110, 513-520). As a result, in plants most gene expression-repressing plant microRNAs function to guide target RNA cleavage (Jones-Rhoades M W and Bartel D P (2004) Mol. Cell 14, 787-799; Kasschau K D et al. (2003) Dev. Cell 4, 205-217). In contrast, most animal microRNAs function to repress expression at the translational or cotranslational level (Ambros V (2003) Cell 113, 673-676; Aukerman M J and Sakai H (2003) Plant Cell 15, 2730-2741; Olsen P H and Ambros V (1999) Dev. Biol. 216, 671-680; Seggerson K et al. (2002) Dev. Biol. 243, 215-225). Although many animal target mRNAs code for developmental control factors, no microRNAs or targets are conserved between plants and animals (Ambros V (2003) Cell 113, 673-676).

In addition to gene expression-repressing microRNAs, plants also produce a second group of expression-regulating RNAs, these are diverse sets of endogenous siRNAs. These differ from microRNAs in that they arise from double-stranded RNA, which requires the activity of RNA-dependent RNA polymerases (RDRs).

Until recently it has been thought that microRNAs and siRNAs in plants and animals function as posttranscriptional negative regulators (Bartel D (2004) Cell 116, 281-297; He L and Hannon G J (2004) Nat. Rev. Genet. 5, 522-531).

Recently, it has been demonstrated in human cells, that small siRNAs and microRNAs targeting the promoter region of a gene are capable of inducing or increasing expression of the respective gene (Li L-C et al. (2006) PNAS 103 (46), 17337-17342; Janowski B A et al. (2007) nature chemical biology 3, 166-173; Place R F et al. (2008) PNAS 105 (5), 1608-1613).

Only few patents have been published claiming use of small RNAs for increase of gene expression. US 2005/0226848 discloses the use of dsRNA molecules for modulating expression of genes in a mammalian in vitro cellular system whereby the modulation comprises increase of gene expression; WO 07/086,990 describes increase of target gene expression in mammalian cells by contacting the cells with oligomeres of 12-28 bp complementary to a promoter region of said target gene; WO 06/113246 describes small activating RNA molecules and their use in mammalian cells. All the applications mentioned claim the use of small activating RNA molecules in animal cells. No such application in plants is suggested.

The mechanism of small RNA mediated activation—increase and/or induction—of gene expression (RNAa) is not yet understood. Place et al. (2008) show for mammalian, that complementarity of small RNA sequence to the targeted DNA sequence is required for function and that RNAa causes changes in chromatin. They speculate that binding of the small RNAs to the respective complementary DNA sequence is necessary for RNAa and that in this regard, the small RNAs function like transcription factors targeting complementary motifs in gene promoters. Another model, discussed by the authors is that the cells may be producing RNA copies of the target promoter region repressing gene expression. By interaction of the complementary microRNAs with the promoter transcripts gene expression is induced or enhanced.

Shibuya et al. (2009) have demonstrated increase of expression of a plant gene, pMADS3, targeted by 100 to 1000 bp dsRNAi constructs directed to an intron of said gene. DsRNAi molecules are inducing a mechanism leading to the generation of 21- to 24 siRNA nucleotide molecules from the precursor involving a set of proteins distinct from that involved for example in processing microRNAs. The siRNA molecules derived from a larger dsRNA molecule are generated randomly and hence a pool of siRNAs differing in their nucleotide sequence is produced from one dsRNA molecule. Shibuya and colleagues showed that methylation of pCG elements in the intron targeted by the dsRNA molecule occurs and speculate that the siRNA molecules derived from the dsRNA molecule trigger methylation in the homologous DNA sequence which leads to induction of expression of the pMADS3 gene. The authors state that the mechanism they observed is different from the RNAa mechanism observed in human cells as histone modification was found in the latter case instead of DNA methylation. They conclude that the mechanism of regulation of gene expression by dsRNAi molecules in plants is distinct from the RNAa mechanism observed in human cells.

In contrast to the observation of increase of gene expression by targeting a regulatory intron with dsRNA molecules in plants, Aufsatz et al (2002) demonstrate gene silencing when promoter sequences are targeted by dsRNA molecules in plants. They show that DNA methylation is involved in this mechanism and that all C residues in the promoter region are methylated that have sequence identity with the dsRNA.

The mechanism of gene expression regulation by small RNAs is distinct between microRNAs and siRNAs. They involve different proteins and cause different effects on DNA, histones and chromatin. Moreover, proteins involved and mechanisms observed differ between animals and plants making it impossible to deduct from observations found in one species to another.

There is a constant need in plant biotechnology for precise increase, induction and/or activation of expression of genes in plants. Methods available so far as the use of promoters and enhancers often lack specificity and/or expression is not strong enough for certain applications. This need is fulfilled with the application at hand.

Surprisingly we observed that the introduction of small double-stranded nucleic acid molecules having homology to a plant specific promoter, for example ta-siRNAs or microRNAs, into plant cells can result in the increase of gene expression of the respective gene operably linked to said promoter. Shibuya et al. (2009) showed that in plants 100 to 1000 bp dsRNA molecules targeting an intron can result in an increase of gene expression by a mechanism which involves the methylation of said intron.

Increase of gene expression of plant genes by introducing small RNA molecules directed to a promoter into a plant or part thereof was not shown before.

A first embodiment of the invention comprises a method for increasing the expression of a target gene in a plant or part thereof, comprising introducing into said plant or part thereof a recombinant nucleic acid molecule not occurring in a respective wild-type plant or part thereof wherein at least a part of said recombinant nucleic acid molecule is complementary to at least a part of a plant specific promoter regulating expression of a target gene in said plant or part thereof and wherein said recombinant nucleic acid molecule confers an increase of expression of said target gene compared to a respective plant or part thereof not comprising said recombinant nucleic acid molecule. It is to be understood that said recombinant nucleic acid molecules may be complementary to either the sense or the antisense strand of at least a part of said plant specific promoter.

The part of said recombinant nucleic acid molecule being complementary to a part of a target gene promoter may be totally complementary or may comprise mismatches. Preferentially, said complementary region comprises 5 or less, 4 or less, 3 or less, 2 or less or 1 mismatches. In an especially preferred embodiment, said complementary region comprises no mismatches and is totally complementary to a part of the target gene promoter. The mismatches are in a preferred embodiment of the invention not localized at any of the positions 4, 5, 6, 16, 17 and/or 18 of the nucleic acid molecule.

The observation of increase of gene expression in plants when targeting the respective promoter with a recombinant nucleic acid molecule being homologous to said promoter is in contrast to the findings that have been published before showing only repression of gene expression in plants when the promoter is targeted by a recombinant nucleic acid molecule (Aufsatz et al (2002)). Although increase of gene expression when targeting the respective promoter with a recombinant nucleic acid has been reported in human cells before this finding was unexpected as mechanisms of gene regulation by small RNAs differ between animal and plant systems (Vaucheret, 2006). The only finding of increased gene expression in plants mediated by small RNAs so far has been the targeting of a regulatory intron in petunia (Shibuya et al. (2009)). Introns are part of the transcribed portion of a gene and are spliced from the pre-mRNA post transcription to generate the mRNA not comprising introns. Promoters in contrast are regulating gene expression and are not transcribed themselves.

The method of the invention for increasing target gene expression in a plant or part thereof comprises introduction of RNA molecules that are homologous to the promoter of a target gene into said plant or part thereof. The introduction could for example be achieved by transient expression of said RNA molecules from vectors that have been introduced in said plant, by introduction of synthesized RNA molecules into the plant cells or by stable transformation of recombinant constructs expressing such RNA molecules or precursors thereof into the genome of plant cell.

The increased expression of a target gene that may be achieved by applying the method of the current invention comprises for example an increase of expression of the target gene in the same tissue/s, developmental stage/s and/or under the same condition/s as the expression of the respective target gene regulated by the respective promoter in a plant or part thereof not comprising the recombinant nucleic acid of the invention. In that way, the expression of a gene which is for example only weakly expressed in a wild-type plant can be increased. This increase expression may have a desirable effect such as for example improved plant health, enhanced yield, increased resistance to biotic or abiotic stress or improved quality of the harvested plant or part thereof. Increased expression may also mean that a target gene is expressed in tissues, at developmental stages or under conditions it is not expressed in a wild-type plant. For example, by applying the method of the invention, an endogenous gene which is only expressed upon infection with a pathogen might be expressed constitutively thereby rendering the plant resistant to said pathogen. The method of the invention may also be used to induce expression of an endogenous gene in a tissue or developmental stage it is not expressed in a wild type plant. The method of the present invention can also be applied to express a transgenic target gene in a plant more precisely. The number and specificity of plant specific promoters available in the art is limited and a promoter having a certain specificity and strength might not always be available. The identification of promoters of such specificity for example tissue specificity is time-consuming and it not always possible for a skilled person to identify such promoter at all. A combination of different promoter specificities known in the art may be needed. The present invention allows increasing target gene expression in all tissues, developmental stages and/or conditions in a plant at which the recombinant nucleic acid molecule is introduced. In one embodiment, such recombinant nucleic acid molecule may be expressed in the plant or part thereof upon transient or stable transformation. Depending on the specificity of the promoter regulating the expression of said recombinant nucleic acid molecule, the target gene expression is increased in those tissues, developmental stages or conditions in which the recombinant nucleic acid is expressed. Thereby the specificities of two promoters may be combined, the one regulating expression of the target gene and the other regulating the expression of the recombinant nucleic acid of the invention targeting the promoter of the target gene. The method is not limited to the combination of the specificity of two promoters as more than one recombinant nucleic acid targeting the same promoter regulating the expression of a target gene may be introduced into a plant or part thereof.

In one embodiment of the invention the recombinant nucleic acid molecule being totally pr partially complementary to at least a part of a promoter regulating expression of a target gene may be complementary to a part of said promoter which is 100 bp or less away of the transcription initiation site. The recombinant nucleic acid may for example be totally or partially complementary to a part of the promoter not more than 100 bp upstream or 100 bp downstream of the transcription initiation site of the promoter. Preferably the recombinant nucleic acid is totally or partially complementary to a part of the promoter which is not more than 50 bp, more preferably not more than 20 bp, even more preferably not more than 10 bp away from the transcription initiation site of the promoter. In a most preferred embodiment of the method of the invention, the recombinant nucleic acid totally or partially complementary to at least a part of a promoter regulating expression of a target gene comprises a complement of the transcription initiation site of said promoter.

It is another embodiment of the present invention, that the recombinant nucleic acid molecule being totally or partially complementary to at least a part of a promoter regulating expression of a target gene is totally or partially complementary to a part of the promoter which is at least 100 bp away of a regulatory box of said promoter. Preferably the recombinant nucleic acid is totally or partially complementary to a part of the promoter which is at least 50 bp, more preferably at least 20 bp, even more preferably at least 10 bp or 5 bp away from a regulatory box of said promoter. In a most preferred embodiment of the method of the invention, the recombinant nucleic acid is totally or partially complementary to a part of the promoter which comprises at least a part of or such regulatory box. Examples of how the present invention may be conducted are given in the examples below. For examples, small synthesized dsRNA molecules of 21 bp increasing target gene expression can be introduced in plant protoplasts. Another example for how to carry out the method of the invention as shown in the examples is the cloning of recombinant pre-miRNAs or ta-siRNAs in which microRNAs or phase regions respectively being homologous to the promoter of a target gene said microRNAs or phase regions increase target gene expression upon processing of the precursor molecules are introduced. The present invention may also be conducted by introduction of long, such as 100 to 1000 bp, dsRNA molecules comprising on the double-stranded stem of the dsRNA molecule regions homologous to the promoter of a target gene and which release upon processing small non coding activating RNAs. Another method may be the expression of short hairpinRNAs from constructs under control of a Pol III RNA gene promoter as for example described in Lu et al. (2004). These recombinant constructs can be transiently or stably transformed into plants or parts thereof generating upon expression and processing RNA molecules homologous to the promoter of a target gene that increase target gene expression. The person skilled in the art is aware of a multitude of other strategies to carry out the present invention.

The recombinant nucleic acid molecule could be introduced into the plant or part thereof using various techniques known to the skilled person. For example, the recombinant nucleic acid molecule can be stable or transiently introduced. Stable introduction could be done for example by transformation using for example Agrobacterium mediated transformation or particle bombardment. The latter could also be used for transient introduction of the recombinant nucleic acid molecules. Other methods for transient introduction of the recombinant nucleic acid molecule of the invention are for example vacuum infiltration, electroporation, chemical induced introduction, the use of viruses or virus derived vectors. The person skilled in the art is aware of other methods useful in the present invention. Preferred methods for the introduction of recombinant nucleic acid molecules in plants or parts thereof are Agrobacterium mediated transformation, particle bombardment, electroporation or chemical induced introduction using for example polyethylene glycol. Especially preferred is Agrobacterium mediated transformation.

Another embodiment of the present invention is a method for increasing the expression of a target gene in a plant or part thereof as described above comprising the steps of

a) producing one or more small nucleic acid molecule complementary to a promoter of a target gene,

b) testing said one or more small nucleic acid molecule in vivo and/or in vitro for their target gene expression increasing property,

c) identifying whether the small nucleic acid molecule increases the target gene expression and

d) introducing said one or more activating small nucleic acid molecule into a plant.

The nucleic acid molecule being complementary to a part of a target gene promoter may be totally complementary or may comprise mismatches. Preferentially, said complementary region comprises 5 or less, 4 or less, 3 or less, 2 or less or 1 mismatches. In an especially preferred embodiment, said complementary region comprises no mismatches and is totally complementary to a part of the target gene promoter. The mismatches are in a preferred embodiment of the invention not localized at any of the positions 4, 5, 6, 16, 17 and/or 18 of the nucleic acid molecule.

The method of the invention as defined above comprises in a first step the screening of small nucleic acid molecules being homologous to the promoter of a target gene for their ability to increase gene expression of said target gene. Said small nucleic acid molecules may be delivered to the plant or part thereof as synthesized small RNA molecules, for example 21 bp double-stranded RNA molecules, or as another example by means of recombinant pre-miRNAs comprising at least one microRNA being homologous to the promoter of a target gene. Upon introduction of the small nucleic acid molecules into the plant or part thereof, the expression of the respective target gene may be analyzed using methods known to the skilled person. The expression may be compared to the expression of the target gene before delivering the small nucleic acid molecules in said plant or part thereof or to a respective wild type plant or part thereof. As an example, the expression of the gene of interest may be analyzed. In another embodiment the promoter of the target gene may be isolated, fused to a reporter gene and introduced in the plant or part thereof prior to screening for small nucleic acid molecules able to increase expression directed by said promoter.

The one or more small nucleic acid molecule being able to increase target gene expression may be used for targeted increase of gene expression of the respective target gene in a method of the invention as described above.

The small nucleic acid molecules can be double-stranded or single-stranded; they may for example consist of DNA and/or RNA oligonucleotides. They can moreover comprise or consist of functional derivatives thereof such as for example PNA. In a preferred embodiment the small nucleic acid molecules are RNA oligonucleotides. In a more preferred embodiment, the RNA oligonucleotides are double-stranded. The length of such oligonucleotides may for example be between about 15 and about 30 bp, for example between 15 and 30 bp, more preferred between about 19 and about 26 bp, for example between 19 and 26 bp, even more preferred between about 20 and about 25 bp for example between 20 and 25 bp. In an especially preferred embodiment the oligonucleotides are between about 21 and about 24 bp, for example between 21 and 24 bp. In a most preferred embodiment, the oligonucleotides are about 21 bp and about 24 bp, for example 21 bp and 24 bp.

The sequences of the small nucleic acid molecules may be totally or partially complementary to one or both strands of the sequence of the promoter. Preferentially they are totally or partially complementary to the sense strand of the sequence of a promoter of a target gene. The sequences of the small nucleic acid molecules may cover the entire sequence of the promoter or parts thereof. The sequence of the small nucleic acid molecules may be overlapping whereby the sequence may be shifted by at least one by or may be adjacent to another without sequence overlap. In a preferred embodiment the small nucleic acid molecules have overlapping sequences shifted by 5 or more, more preferable by 3 or more and even more preferable by 1 bp or more.

The small nucleic acid molecule may be introduced into a plant or part thereof individually or in pools. They may for example be introduced by means of electroporation or chemically mediated transformation into protoplasts. Alternatively, the small nucleic acid molecules may be tested in vitro in cell free systems. Small nucleic acid molecules increasing the expression of the respective target gene may for example be identified by analyzing the expression of said target gene before and after introduction of the small nucleic acid molecules into the cell or cell free system with methods known to the skilled person. Once a small nucleic acid molecule increasing the respective target gene is identified, this small nucleic acid molecule may be used for directed increase of expression of the respective target gene by introducing said small nucleic acid molecule into a plant or part thereof.

A further embodiment of the invention is a method for increasing the expression of a target gene in a plant or part thereof as described above wherein said small nucleic acid molecule increasing the target gene is introduced into said plant by cloning the small nucleic acid molecule increasing the target gene into a plant transformation vector comprising a plant specific regulatory element, transforming a plant or parts thereof with said vector and recovering a transgenic plant comprising said vector or a part of said vector such as the T-DNA region.

As described above, small activating nucleic acid molecules can transiently be introduced into a plant or part thereof or they may be expressed from nucleic acid constructs that are stable integrated into the genome of a plant or part thereof. In the latter case, the skilled person is aware of methods of how to produce chimeric recombinant constructs directing expression in plants or parts thereof. For example, the small nucleic acid molecule can be cloned by recombinant DNA techniques into plant transformation vectors. For example, the small nucleic acid molecule activating gene expression of a target gene might be introduced into a microRNA gene or ta-siRNA gene replacing at least one phase region in the ta-siRNA gene. Replacing as meant herein means the addition of a phase region or microRNA in the respective gene, the substitution of the endogenous microRNA or phase region with another microRNA or phase region. It can also mean the mutation of the sequence of a microRNA or phase region by for example exchanging, deleting or inserting a base pair.

Such genes when expressed in a plant cell or part thereof are forming RNA precursor molecules comprising the recombinant region homologous to a plant specific promoter. The precursor molecule might subsequently be processed releasing the recombinant small RNA molecule homologous to a target gene promoter. Additional genetic elements might be present on said vector such as a promoter controlling expression of the small nucleic acid molecule or the respective precursor molecules. Other genetic elements that might be comprised on said vector might be a terminator. Methods for introducing such a vector comprising such an expression construct comprising for example a promoter, said small nucleic acid molecule and a terminator into the genome of a plant and for recovering transgenic plants from a transformed cell are also well known in the art. Depending on the method used for the transformation of a plant or part thereof the entire vector might be integrated into the genome of said plant or part thereof or certain components of the vector might be integrated into the genome, such as, for example a T-DNA.

A further embodiment of the invention relates to a method for increasing the expression of a target gene in a plant or part thereof, comprising introducing into said plant or part thereof a recombinant nucleic acid molecule encoding a modified small non-coding RNA (sncRNA), wherein the sequence of said sncRNA is modified in relation to a natural sncRNA sequence by replacing at least one region of said natural sncRNA complementary to its respective natural target sequence by a sequence, which is complementary to a plant specific promoter regulating expression of a target gene and which is heterologous with regard to said natural sncRNA.

The part of said recombinant nucleic acid molecule being complementary to a part of a target gene promoter may be totally complementary or may comprise mismatches.

Preferentially, said complementary region comprises 5 or less, 4 or less, 3 or less, 2 or less or 1 mismatches. In an especially preferred embodiment, said complementary region comprises no mismatches and is totally complementary to a part of the target gene promoter. The mismatches are in a preferred embodiment of the invention not localized at any of the positions 4, 5, 6, 16, 17 and/or 18 of the nucleic acid molecule.

The invention could for example be carried out by isolating a sncRNA gene. SncRNA genes that can be used in the method of the invention are known to a skilled person. A sncRNA gene may comprise regions being homologous to the natural target gene of said sncRNA gene. Such region can be replaced by a sequence being homologous to the promoter of a target gene wherein the replacing sequence is known to increase gene expression of the target gene when a nucleic acid molecule of the respective sequence is introduced into a plant cell. Methods for replacing a region in an isolated nucleic acid molecule are known to a skilled person. Upon introduction into a plant or part thereof such modified sncRNA gene is expressed into a precursor RNA molecule comprising a region homologous to a target gene promoter. The precursor molecule is subsequently processed thereby releasing one or more small double-stranded regulatory RNA molecule of for example 21 or 24 bp length being homologous to the promoter of a target gene. These small double-stranded regulatory RNA molecules are triggering increase of expression of said target gene. Natural small non coding regulatory RNAs are for example comprised on precursor molecules encoded in the genome. Such small non coding regulatory RNAs are for example microRNAs or ta-siRNAs. Other sncRNAs may be for example shRNAs, snRNAs, nat-siRNA and/or snoRNAs. Preferred sncRNAs are ta-siRNAs, nat-siRNAs and microRNAs. Especially preferred are microRNAs.

These precursor molecules are recognized in the plant cell by a specific set of proteins that process these precursor molecules thereby releasing the small regulatory RNAs such as miRNAs or siRNAs. The processing of such precursor molecules releases single stranded or double-stranded RNA molecules of for example 21 or 24 bp length of defined sequence. Other precursor molecules such as large hairpin dsRNA molecules are processed by proteins releasing for example 21 or 24 bp dsRNA molecules of random sequence. The different plant pathways for processing precursor sncRNAs are for example described in Vaucheret (2006).

A person skilled in the art is aware of methods of how to modify or synthesize such genes of precursor molecules releasing small non coding activating RNA molecules homologous to the promoter of a target gene.

A phase region comprised on a ta-siRNA which is released from said ta-siRNA upon processing of the same may be replaced by methods known in the art such as cloning techniques or recombination or the entire ta-siRNA comprising a phase region directed to a promoter might be synthesized in vitro. In a preferred embodiment, all phase regions of a natural ta-siRNA are replaced by sequences totally or partially complementary to a plant specific promoter regulating the expression of a target gene. For example, the sequences replacing the phase regions in a natural sncRNA might all be totally or partially complementary to the same plant specific promoter regulating the expression of a target gene. Alternatively, the sequences replacing the phase regions in a natural ta-siRNA might be totally or partially complementary to different plant specific promoters regulating the expression of one target gene or to different plant specific promoters regulating the expression of different target genes.

In another embodiment, a pre-miRNA might be employed for activating the expression of a target gene in a plant or part thereof. Methods for replacing a microRNA comprised on a pre-miRNA molecule are known in the art and are for example described in Schwab R et al. (2006) Highly Specific Gene Silencing by Artificial MicroRNAs in Arabidopsis Plant Cell 18: 1121-1133.

A further embodiment of the invention is a method for identifying small non-coding activating RNA (sncaRNA) molecules in a plant or part thereof comprising the steps of obtaining small RNA molecules from said plant or part thereof, identifying the sequence of said small RNA molecules selecting small RNA molecules comprising regions complementary to at least one promoter of an endogenous gene via bioinformatic analysis and testing small RNA candidates in a plant or part thereof to determine increase of gene expression triggered by small RNA molecules.

Methods for obtaining small RNA molecules and the respective sequences from biological material such as a plant or part thereof are described in the art (Sunkar R and Zhu J (2004) Novel and stress-regulated microRNAs and other small RNAs from Arabidopsis. The Plant Cell 16:2001-2019; Lu C et al (2005) Elucidation of the small RNA components of the transcriptome. Science 309:1567-1569).

These methods can for example be applied in the present invention. The sequences can be analyzed for homology to at least one promoter of an endogenous gene using bioinformatic tools such as described in Jones-Rhoades M and Bartel D (2004) Computational identification of plant microRNAs and their targets, including a stress-induced miRNA. Molecular Cell 14: 787-799; Zhang Y (2005) miRU: an automated plant miRNA target prediction server; Griffiths-Jones S et al (2006) miRBase: microRNA sequences, targets and gene nomenclature Nucleic Acids Research 34:D14-D144 or for example Johnson C et al (2006) CSRDB: a small RNA integrated database and browser resource for cereals D1-D5.

The sequences obtained as described above can be analyzed for homology to any promoter sequence information of the plant, the small RNA sequences were obtained from. It is also possible to search the identified small RNA sequences for homology to one or more specific target gene promoter sequences. The small RNA sequences found to be homologous to a promoter sequence of an endogenous gene may in a further step be tested for their ability of regulating increase of gene expression. Methods useful for such testing are for example described above and comprise introduction of the identified small RNA sequences into in vivo or in vitro test systems, analyzing in said test system the expression of the respective target gene, a set of potential target genes or the expression of all genes. For example, the expression of a specific gene or a set of genes may be tested with northern blot or qPCR. The expression of a large set or all genes of a plant or part thereof may be tested using chip hybridization or equivalent methods known to a skilled person.

An additional embodiment of the invention is a method for identifying activating microRNAs in a plant or part thereof comprising the steps of identifying microRNAs in said plant or part thereof being homologous to a sequence of a promoter in the respective plant, cloning said microRNAs from said plant or part thereof, introducing said microRNAs in a plant and comparing gene expression of potential target genes in said plants comprising said microRNA with respective wild-type plants.

MicroRNAs as meant herein are RNA molecules that are 18 to 24 nucleotides in length, which regulate gene expression. MicroRNAs are encoded by non protein coding genes that are transcribed into a primary transcript which is forming a stem-loop structure called a pre-miRNA. The microRNA is processed from said pre-miRNA and released as double stranded RNA molecule.

Methods for identifying microRNAs from biological material such as a plant are described in the art (Sunkar R and Zhu J (2004) Novel and stress-regulated microRNAs and other small RNAs from Arabidopsis. The Plant Cell 16:2001-2019 and Lu C et al (2005) Elucidation of the small RNA components of the transcriptome. Science 309:1567-1569). These methods can for instance be applied in this embodiment of the present invention. The microRNA region of these pre-miRNAs can be determined as described in the art and tested with bioinformatic tools for homology to plant specific promoters in the plant the microRNAs were derived from. Bioinformatic tools that can be applied in that analysis are known in the art and examples are given above. In order to test for gene expression increasing activity of the microRNAs identified, said microRNAs might be synthesized and introduced for example into a plant cell, protoplast or cell free system. The gene expression increasing activity of said microRNAs might also be tested by cloning and over expressing the respective microRNA encoding gene. Methods for cloning and over expression of microRNAs are for example described in Schwab R et al. (2006) Highly Specific Gene Silencing by Artificial MicroRNAs in Arabidopsis Plant Cell 18: 1121-1133 or Warthmann N et al (2008) Highly Specific Gene Silencing by Artificial miRNAs in Rice PLoS ONE 3(3): e1829.

It is also an embodiment of the present invention to isolate such sncaRNA encoding genes, for example activating microRNA encoding genes and introduce them in plants or parts thereof in order to increase expression of the respective target genes. The sncaRNA encoding gene, for example activating microRNA encoding gene may for example be operably linked to a heterologous promoter. Such recombinant construct may be comprised on a vector and transformed into a plant or part thereof. The heterologous promoter regulating expression of said sncaRNA encoding gene may confer expression of the sncaRNA in tissues, developmental stages and/or under conditions such as for example stress conditions as drought or cold, the sncaRNA is not expressed in a reference plant, for example a wild-type plant, not comprising the respective construct. Thereby expression of the respective target gene in the plant in said tissue, developmental stage and/or condition is increased or induced.

A method for replacing the regulatory specificity of a plant specific promoter by modifying in said plant specific promoter a sector targeted by a sncRNA conferring increase of expression of genes controlled by said promoter is a further embodiment of the present invention.

“Replacing the regulatory specificity” as understood here means that the regulatory specificity of a promoter adapted according to the invention differs from the regulatory specificity of the promoter before the method of the invention has been applied. The regulatory specificity may be differing in expression strength meaning that the adapted promoter is conferring expression for example in the same tissues, developmental stage and or conditions but the expression is higher compared to the promoter before the method of the invention has been applied to the promoter. It may also mean that the promoter confers expression in additional or other tissues, cells, compartments of the plant, in additional or other developmental stages of the plant or under additional or different conditions such as environmental conditions compared to the promoter before the method of the invention has been applied.

The specificity of a promoter is amongst others depending on its DNA sequence at the interaction with various proteins and RNA molecules. The interaction with said RNA molecules also depends on sequence of the promoter. Hence, it is possible to change the specificity of a promoter by changing the sequence of at least on of those sectors on the promoter that are necessary for interaction with regulatory RNAs. These sectors could for example be modified by conversion of the sequence, deletion or insertion in a way that the endogenous sncRNA, interacting with said sector is not longer able to interact. This could for example lead to a down-regulation of the promoter in certain tissues or developmental stages in case the interacting RNA had been a sncaRNA. The sector sequence may also be adapted in way that another sncRNA is interacting with that sector leading to a change in the specificity of the promoter.

The present invention also relates to a method for replacing the regulatory specificity of a plant specific promoter by introducing into said plant specific promoter a sector targeted by a recombinant sncaRNA conferring increase of expression of genes controlled by said promoter and wherein the recombinant sncaRNA is under control of a plant specific promoter conferring an increase of expression of the target gene according to the specificity of the plant specific promoter controlling the sncaRNA.

The specificity of a promoter may according to the present invention be changed by introducing into the promoter sequence a new sector interacting with a recombinant sncaRNA that will be introduced in the plant or part thereof comprising said promoter. The introduction may be an insertion leading to an increased length of the promoter or a replacement for a sequence of similar or same size as the introduced sector keeping the sequence length of the promoter substantially unchanged.

Modifying a sector as used herein means for example replacing a sector targeted by an sncRNA by another one, targeted by a sncaRNA or mutating the sequence of a sector in a way, that a sncaRNA is targeting it or in a way, that the sector is not targeted any more by the endogenous regulatory small RNA that has been targeted the sector before. It may also mean deleting a sector from the plant specific promoter. Deleting a sector could mean deleting the sector and fusing the DNA strands that were adjacent to said sector or by replacing the sector by a random DNA molecule of about the same size as the sector, said DNA molecule being not targeted by a sncRNA. In the first case, the promoter sequence is shorter after deleting the sector, in the latter case, the promoter sequence has about the same size as it had before the sector had been deleted. Irrespective of how the deletion of the sector is done, the sncRNA is no longer able to interact with the so modified plant specific promoter.

It is also one embodiment of the present invention to replace the regulatory specificity of a plant specific promoter by introducing into said plant specific promoter a sector targeted by an endogenous sncaRNA conferring increase of expression of genes controlled by said promoter.

For example, the method of modifying the regulatory specificity of a plant specific promoter could be employed such that the at least one sector introduced into the plant specific promoter is replacing a sector targeted by an endogenous sncRNA. The at least one sector replacing said endogenous sector targeted by an endogenous sncaRNA could itself be targeted by another endogenous sncaRNA with a differing specificity as the sncRNA that has targeted the endogenous sector or by a recombinant sncaRNA introduced in the respective plant or part thereof.

The modification of the plant specific promoter could in one embodiment of the invention be done in vivo by for example applying recombination techniques. The plant specific promoter sequence in this embodiment may be modified while it is in the genome of a viable cell or an intact cell compartment. While and after applying these techniques the plant specific promoter to be modified is kept in its original genomic context. In another embodiment of the invention, the plant specific promoter may be isolated from its natural context and the regulatory region may be modified in vitro by techniques known in the art for example recombinant DNA techniques like cloning techniques, recombination or synthesis. The at least one sector to be modified in a plant specific promoter might as well be modified by mutating its original sequence. For example, at least one base pair might be exchanged in the sequence of the sector, or at least one base pair might be deleted or introduced. As a result of such mutation the at least one sector might not longer be targeted by the sncRNA that had targeted said sector before, hence it might not longer be targeted by a sncRNA at all or might be targeted by another sncaRNA, which might be endogenous or recombinant. The regulatory specificity of a plant specific promoter could also be modified by deleting at least one sector targeted by an endogenous sncaRNA from said regulatory sequence. The sector may be deleted completely or in part, in vitro or in vivo as described above.

The introduction of a sector targeted by a recombinant sncaRNA into a plant specific promoter can be achieved by inserting a sector into said promoter thereby extending the length of said regulatory region, by replacing a part of said regulatory region for example replacing an endogenous sector targeted by an endogenous sncaRNA or by mutating the sequence of said regulatory region. As pointed out above, the respective methods might be applied in vivo or in vitro. Alternatively, the entire plant specific promoter molecule might be synthesized by methods known in the art.

The recombinant sncaRNA introduced into the plant might target specifically one target gene or several target genes that should be coordinately activated in a plant or part thereof. Replacing the regulatory specificity of a plant specific promoter comprises for example the activation of a plant specific, for example plant tissue specific promoter having a desirable specificity but is not generating an expression rate as needed. Such promoter could be specifically activated by introducing into said promoter a sector targeted by a recombinant sncaRNA being under control of a promoter leading to expression of said recombinant sncaRNA in the tissue where an increased activity of the target gene is desirable. Replacing the regulatory specificity of a plant specific promoter might also mean activation of a promoter in for example a tissue or developmental stage in which it normally is not active. Moreover the method could be useful to repress the activity of a promoter for example in a tissue or developmental stage by increasing a repressor gene targeting the gene of interest, thereby improving the specificity of a given regulatory sequence.

A nucleic acid construct for expression in plants comprising a recombinant nucleic acid molecule comprising a sequence encoding a modified small non-coding RNA (sncRNA) sequence, wherein said sequence is modified in relation to a wild-type sncRNA sequence by at least replacing one region of said wild-type sncRNA complementary to its respective wild-type target sequence by a sequence, which is complementary to a plant specific promoter regulating expression of a target gene and which is heterologous with regard to said wild-type sncRNA and which confers increase of expression of said target gene upon introduction into said plant or part thereof is also an embodiment of the present invention. The sequence complementary to a plant specific promoter may be totally complementary or may comprise mismatches. Preferentially, said complementary sequence comprises 5 or less, 4 or less, 3 or less, 2 or less or 1 mismatches. In an especially preferred embodiment, said complementary sequence comprises no mismatches and is totally complementary to a part of the target gene promoter. The mismatches are in a preferred embodiment of the invention not localized at any of the positions 4, 5, 6, 16, 17 and/or 18 of the complementary sequence.

In a further embodiment the transcript of the recombinant nucleic acid molecule comprised in the nucleic acid construct as described above is able to form a double stranded structure, wherein said double stranded structure comprises the sequence being complementary to a plant specific promoter regulating expression of a target gene.

In a preferred embodiment the double stranded structure is a hairpin structure.

It is also an embodiment of the present invention that the part of the recombinant nucleic acid molecule being complementary to a plant specific promoter regulating expression of a target gene as described above has a length for example from about 15 to about 30 bp, for example from 15 to 30 bp, preferably about 19 to about 26 bp, for example from 19 to 26 bp, more preferably from about 21 to about 25 bp, for example from 21 to 25 bp, even more preferably 21 or 24 bp.

The part of said recombinant nucleic acid molecule being complementary to a plant specific promoter regulating expression of a target gene comprised on the nucleic acid construct as described above might have an identity of 60% or more, preferably 70% or more, more preferably 75% or more, even more preferably 80% or more, most preferably 90% or more.

Said recombinant nucleic acid molecule being complementary to a plant specific promoter regulating expression of a target gene might further comprises at least about 7 to about 11, for example 7 to 11, preferably about 8 to about 10, for example 8 to 10, more preferably about 9, for example 9 consecutive base pairs homologous to said target gene regulatory element.

The said consecutive base pairs are at least 80% identical, preferably 90% identical, more preferably 95% identical, most preferably 100% identical to said target gene regulatory element.

The part of said recombinant nucleic acid molecule being complementary to a part of a target gene promoter may be totally complementary or may comprise mismatches. Preferentially, said complementary region comprises 5 or less, 4 or less, 3 or less, 2 or less or 1 mismatches. In an especially preferred embodiment, said complementary region comprises no mismatches and is totally complementary to a part of the target gene promoter. The mismatches are in a preferred embodiment of the invention not localized at any of the positions 4, 5, 6, 16, 17 and/or 18 of the nucleic acid molecule.

The recombinant nucleic acid molecule being complementary to a plant specific promoter could be comprised for example in a pre-miRNA gene, a gene encoding a ta-sRNA or any other gene able to release small RNA molecules upon expression in a plant or part thereof.

A further embodiment of the present invention is a vector comprising a nucleic acid construct as defined above.

The present invention further provides a system for increasing gene expression in a plant or part thereof comprising

a) a plant specific promoter comprising a sector targeted by a small activating RNA heterologous to said plant specific promoter and

b) a construct comprising a small activating RNA targeting the sector as defined in a) under the control of a plant specific promoter.

A system as described above allows a precise expression of a target gene in a plant or part thereof. The specificity of expression of a target gene is depending on the goal to be achieved with the respective application. For example it might be advantageous to express a target gene in two different tissues or in the same tissue at different developmental stages of a plant. Endogenous promoters having such specificities are often not available and might not even exist. A system as described above may be used to combine the specificities of different promoters by introducing a specific sector into a given promoter targeted by a recombinant sncaRNA. In that way, the expression pattern of two different promoters may be combined as the expression of the recombinant promoter is increased upon the interaction with the sncaRNA molecule expressed by a different promoter having a different specificity. Likewise the sncaRNA molecule might be expressed under the control of the same promoter as the target gene leading to an increased expression of the target gene in the target tissues without altering the expression pattern of the promoter.

Hence the specificity of the expression of a target gene can be adapted to the need of the user.

The system as defined above might for example be applied for increasing gene expression of an endogenous gene. For that purpose, a sncaRNA might be introduced into a plant that is targeting and increasing expression of the endogenous promoter of the target gene. It might also be possible to introduce in the promoter of the endogenous gene a sector targeted by a sncRNA known to increase expression when interacting with a given promoter. The sector may be introduced in the endogenous promoter in vitro or in vivo by recombinant DNA techniques known to the skilled person.

The system might as well be used for increasing gene expression of a transgene. For that purpose a sector targeted by a sncaRNA may be introduced in the sequence of a promoter controlling expression of a transgenic target gene. The construct comprising the recombinant promoter and the target gene may be introduced in a plant or part thereof on the same construct as the gene encoding the respective sncaRNA; the two components might be on distinct constructs and introduced into a plant or part thereof at the same time or in subsequent steps of transformation and/or crossing.

A plant or part thereof, for example a plant cell, comprising a recombinant nucleic acid construct as defined above, wherein said recombinant nucleic acid molecule causes an increase of expression of a target gene in said plant or part thereof compared to a respective plant or part thereof not comprising said recombinant nucleic acid molecule is also enclosed in the present invention.

In one embodiment, said recombinant nucleic acid molecule is integrated into the genome of said plant or part thereof. The genome as meant here includes the nuclear genome, the genome comprised in the plastids of plants, also known as plastome, as well as the genome comprised in the mitochondria of plants.

A further embodiment of the present invention is a method as defined above comprising a nucleic acid construct as defined above, a plant as defined above and/or a plant cell as defined above.

A further embodiment of the present invention is a microorganism which is able to transfer nucleic acids to a plant or part of a plant wherein said microorganism is comprising a recombinant nucleic acid construct as defined above, wherein said recombinant nucleic acid molecule confers upon transfer of said recombinant nucleic acid construct into a plant or part of a plant an increase of expression of a target gene in said plant or part of a plant compared to a respective plant or part of a plant not comprising said recombinant nucleic acid molecule. Such microorganism is preferentially of the genus Agrobacteria, preferentially Agrobacterium tumefaciens or Agrobacterium rhizogenes. In a most preferred embodiment, the microorganism is Agrobacterium tumefaciens.

A method for production of a nucleic acid construct as defined above, a vector as defined above, a plant as defined above and/or a part of a plant or a plant cell as defined in above are further embodiments of the present invention.

Further embodiments of the present invention are sncaRNAs conferring an increase of gene expression in a plant or part thereof comprising the sequence of anyone of SEQ ID 6, 7, 8, 9, 10, 11, 12, 13, 14, 15, 16, 17, 18, 19, 22, 23, 24, 25, 26, 27, 28, 29, 30 and/or 31.

The use of a small non-coding activating RNA as defined above for increasing the expression of a target gene in a plant is also an embodiment of the present invention. The sncaRNA molecules might in that embodiment for example be used for increasing the expression of an endogenous target gene or for increasing the expression of a transgenic target gene.

### DEFINITIONS

Abbreviations: BAP—6-benzylaminopurine; 2,4-D—2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3—Gibberellic acid; Timentin™: ticarcillin disodium/clavulanate potassium.

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Activate: To “activate”, “induce” or “increase” the expression of a nucleotide sequence in a plant cell means that the level of expression of the nucleotide sequence in a plant cell after applying a method of the present invention is higher than its expression in the plant, part of the plant or plant cell before applying the method, or compared to a reference plant lacking the chimeric RNA molecule of the invention. The term “activated”, “induced” or “increased” as used herein are synonymous and means herein higher, preferably significantly higher expression of the nucleotide sequence. “Higher expression” could also mean that the expression of the nucleotide sequence was not detectable before a method of the present invention has been applied. As used herein, an “activation”, “induction” or “increase” of the level of an agent such as a protein or mRNA means that the level is increased relative to a substantially identical plant, part of a plant or plant cell grown under substantially identical conditions, lacking a chimeric RNA molecule of the invention capable of activating the agent. As used herein, “activation”, “induction” or “increase” of the level of an agent (such as for example an preRNA, mRNA, rRNA, tRNA, snoRNA, snRNA expressed by the target gene and/or of the protein product encoded by it) means that the level is increased 10% or more, for example 50% or more, preferably 100% or more, more preferably 5 fold or more, most preferably 10 fold or more, for example 20 fold relative to a cell or organism lacking a chimeric RNA molecule of the invention capable of inducing said agent. It could also mean that the expression of a gene is detectable after application of a method of the present invention, whereas it has not been detectable before said application of said method. The activation, increase or induction can be determined by methods with which the skilled worker is familiar. Thus, the activation, increase or induction of the protein quantity can be determined for example by an immunological detection of the protein. Moreover, biochemical techniques such as Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

Agronomically valuable trait: The term “agronomically valuable trait” refers to any phenotype in a plant organism that is useful or advantageous for food production or food products, including plant parts and plant products. Non-food agricultural products such as paper, etc. are also included. A partial list of agronomically valuable traits includes pest resistance, vigor, development time (time to harvest), enhanced nutrient content, novel growth patterns, flavors or colors, salt, heat, drought and cold tolerance, and the like. Preferably, agronomically valuable traits do not include selectable marker genes (e.g., genes encoding herbicide or antibiotic resistance used only to facilitate detection or selection of transformed cells), hormone biosynthesis genes leading to the production of a plant hormone (e.g., auxins, gibberllins, cytokinins, abscisic acid and ethylene that are used only for selection), or reporter genes (e.g. luciferase, glucuronidase, chloramphenicol acetyl transferase (CAT, etc.). Such agronomically valuable important traits may include improvement of pest resistance (e.g., Melchers et al. (2000) Curr Opin Plant Biol 3(2):147-52), vigor, development time (time to harvest), enhanced nutrient content, novel growth patterns, flavors or colors, salt, heat, drought, and cold tolerance (e.g., Sakamoto et al. (2000) J Exp Bot 51(342):81-8; Saijo et al. (2000) Plant J 23(3): 319-327; Yeo et al. (2000) Mol Cells 10(3):263-8; Cushman et al. (2000) Curr Opin Plant Biol 3(2):117-24), and the like. Those of skill will recognize that there are numerous polynucleotides from which to choose to confer these and other agronomically valuable traits.

Amino acid sequence: As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Conferring activation of expression as used herein means that upon interaction of a peptide, protein and/or nucleic acid molecule, for example a RNA molecule with the promoter of a gene the expression of said gene is increased, induced or activated compared to the expression of said gene before interaction of the promoter of said gene with said peptide, protein and/or nucleic acid molecule. The interaction of the promoter with the peptide, protein and/or nucleic acid molecule, for example a RNA molecule may be a direct interaction, for example binding or an indirect interaction, whereby said peptide, protein and/or nucleic acid molecule involve further elements in order to confer activation of expression.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule. Preferably the terms refer to a double-stranded RNA molecule capable, when introduced into a cell or organism, of causing or increasing the level of an mRNA species present in a cell or a cell of an organism.

As used herein, “RNA activation”, “RNAa”, and “dsRNAa” refer to gene-specific increase of expression that is induced by a RNA molecule, for example a double-stranded RNA molecule. Said RNA molecule might be an endogenous RNA molecule or introduced into a plant or part thereof for example as dsRNA molecule or comprised on a construct producing said RNAa molecules upon expression. The double-stranded RNA molecule might for example be a microRNA or a ta-siRNA.

Endogenous: An “endogenous” nucleotide sequence refers to a nucleotide sequence, which is present in the genome of the untransformed plant cell.

Essential: An “essential” gene is a gene encoding a protein such as e.g. a biosynthetic enzyme, receptor, signal transduction protein, structural gene product, or transport protein that is essential to the growth or survival of the plant or plant cell.

Expression: “Expression” refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, or any other noncoding regulatory RNA, in the sense or antisense direction. The expression construct comprising the nucleotide sequence of interest may be chimeric, meaning that at least one of its components is heterologous with respect to at least one of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Hairpin: As used herein “hairpin RNA” or “hairpin structure” refers to any self-annealing double stranded RNA or DNA molecule. In its simplest representation, a hairpin structure consists of a double stranded stem made up by the annealing nucleic acid strands, connected by a single stranded nucleic acid loop, and is also referred to as a “pan-handle nucleic acid”. However, the term “hairpin RNA” or “hairpin structure” is also intended to encompass more complicated secondary nucleic acid structures comprising self-annealing double stranded sequences, but also internal bulges and loops. The specific secondary structure adapted will be determined by the free energy of the nucleic acid molecule, and can be predicted for different situations using appropriate software such as FOLDRNA (Zuker and Stiegler (1981) Nucleic Acids Res 9(1):133-48; Zuker, M. (1989) Methods Enzymol. 180:262-288).

Heterologous: The terms “heterologous” with respect to a nucleic acid molecule or DNA refer to a nucleotide sequence which is operably linked to, or is manipulated to become operably linked to, a nucleic acid molecule sequence to which it is not operably linked in nature, or to which it is operably linked at a different location in nature. A heterologous expression construct comprising a nucleic acid sequence and at least one regulatory sequence (such as a promoter or a transcription termination signal) linked thereto for example is a constructs originating by experimental manipulations in which either a) said nucleic acid sequence, or b) said regulatory sequence or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural chromosomal locus in the organism of origin, or to the presence in a genomic library. In the case of a genomic library, the natural genetic environment of the nucleic acid sequence is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid sequence operably linked to a promoter, which is not the native promoter of this sequence, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA and proteins that are not normally produced by the cell into which it is expressed.

Homologous DNA Sequence: “Homologous” when used in respect to the comparison of two or more nucleic acid or amino acid molecules means that the sequences of said molecules share a certain degree of sequence similarity, the sequences being partially identical.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

Low stringency conditions when used in reference to nucleic acid hybridization comprise conditions equivalent to binding or hybridization at 68° C. in a solution consisting of 5×SSPE (43.8 g/L NaCl, 6.9 g/L NaH₂PO₄.H₂O and 1.85 g/L EDTA, pH adjusted to 7.4 with NaOH), 1% SDS, 5×Denhardt's reagent [50×Denhardt's contains the following per 500 mL 5 g Ficoll (Type 400, Pharmacia), 5 g BSA (Fraction V; Sigma)] and 100 μg/mL denatured salmon sperm DNA followed by washing (preferably for one times 15 minutes, more preferably two times 15 minutes, more preferably three time 15 minutes) in a solution comprising 1×SSC (1×SSC is 0.15 M NaCl plus 0.015 M sodium citrate) and 0.1% SDS at room temperature or—preferably 37° C.—when a DNA probe of preferably about 100 to about 1,000 nucleotides in length is employed.

Medium stringency conditions when used in reference to nucleic acid hybridization comprise conditions equivalent to binding or hybridization at 68° C. in a solution consisting of 5×SSPE (43.8 g/L NaCl, 6.9 g/L NaH₂PO₄.H₂O and 1.85 g/L EDTA, pH adjusted to 7.4 with NaOH), 1% SDS, 5×Denhardt's reagent [50×Denhardt's contains the following per 500 mL 5 g Ficoll (Type 400, Pharmacia), 5 g BSA (Fraction V; Sigma)] and 100 μg/mL denatured salmon sperm DNA followed by washing (preferably for one times 15 minutes, more preferably two times 15 minutes, more preferably three time 15 minutes) in a solution comprising 0.1×SSC (1×SSC is 0.15 M NaCl plus 0.015 M sodium citrate) and 1% SDS at room temperature or—preferably 37° C.—when a DNA probe of preferably about 100 to about 1,000 nucleotides in length is employed.

High stringency conditions when used in reference to nucleic acid hybridization comprise conditions equivalent to binding or hybridization at 68° C. in a solution consisting of 5×SSPE, 1% SDS, 5×Denhardt's reagent and 100 μg/mL denatured salmon sperm DNA followed by washing (preferably for one times 15 minutes, more preferably two times 15 minutes, more preferably three time 15 minutes) in a solution comprising 0.1×SSC, and 1% SDS at 68° C., when a probe of preferably about 100 to about 1,000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence. When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

“Identity”: The term “identity” is a relationship between two or more polypeptide sequences or two or more nucleic acid molecule sequences, as determined by comparing the sequences. In the art, “identity” also means the degree of sequence relatedness between polypeptide or nucleic acid molecule sequences, as determined by the match between strings of such sequences. “Identity” as used herein can be measured between nucleic acid sequences of the same ribonucleic-type (such as between DNA and DNA sequences) or between different types (such as between RNA and DNA sequences). It should be understood that in comparing an RNA sequence to a DNA sequence, an “identical” RNA sequence will contain ribonucleotides where the DNA sequence contains deoxyribonucleotides, and further that the RNA sequence will contain a uracil at positions where the DNA sequence contains thymidine. In case an identity is measured between RNA and DNA sequences, uracil bases of RNA sequences are considered to be identical to thymidine bases of DNA sequences. “Identity” can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J. Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12:76-80 (1994); Birren et al., Genome Analysis, 1:543-559 (1997)). The BLASTX program is publicly available from NCBI and other sources (BLAST Manual, Altschul, S., et al., NCBI NLM NIH, Bethesda, Md. 20894; Altschul, S., et al., J. Mol. Biol., 215:403-410 (1990)). The well-known Smith Waterman algorithm can also be used to determine identity. Parameters for polypeptide sequence comparison typically include the following:
Algorithm: Needleman and Wunsch, J. Mol. Biol., 48:443-453 (1970)
Comparison matrix: BLOSUM62 from Hentikoff and Hentikoff, Proc. Natl. Acad. Sci. USA, 89:10915-10919 (1992)
Gap Penalty: 12
Gap Length Penalty: 4

A program, which can be used with these parameters, is publicly available as the “gap” program from Genetics Computer Group, Madison, Wis. The above parameters along with no penalty for end gap are the default parameters for peptide comparisons. Parameters for nucleic acid molecule sequence comparison include the following:
Algorithm: Needleman and Wunsch, J. Mol. Bio. 48:443-453 (1970)
Comparison matrix: matches-+10; mismatches=0
Gap Penalty: 50
Gap Length Penalty: 3

As used herein, “% identity” is determined using the above parameters as the default parameters for nucleic acid molecule sequence comparisons and the “gap” program from GCG, version 10.2.

Intron: The term “intron” as used herein refers to the normal sense of the term as meaning a segment of nucleic acid molecules, usually DNA, that does not encode part of or all of an expressed protein, and which, in endogenous conditions, is transcribed into RNA molecules, but which is spliced out of the endogenous RNA before the RNA is translated into a protein. The splicing, i.e., intron removal, occurs at a defined splice site, e.g., typically at least about 4 nucleotides, between cDNA and intron sequence. For example, without limitation, the sense and antisense intron segments illustrated herein, which form a double-stranded RNA contained no splice sites. Introns may inhere regulatory function regulating gene expression for example introns may regulate expression specificity or strength or they may influence efficiency of RNA splicing or RNA stability.

“Increase”: the terms “activate”, “increase” and “induce” as used herein in respect to the expression of a gene are used as synonyms. See the definition above for “activate”.

Isogenic: organisms (e.g., plants), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Operable linkage: The term “operable linkage” or “operably linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Operable linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (NY); Silhavy et al. (1984) Experiments with Gene Fusions, Cold Spring Harbor Laboratory, Cold Spring Harbor (NY); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of promoter and nucleic acid sequence to be expressed, can exist in a vector-integrated form and be inserted into a plant genome, for example by transformation.

Organ: The term “organ” with respect to a plant (or “plant organ”) means parts of a plant and may include (but shall not limited to) for example roots, fruits, shoots, stem, leaves, anthers, sepals, petals, pollen, seeds, etc.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Part of a plant: The term “part of a plant” comprises any part of a plant such as plant organs or plant tissues or one or more plant cells which might be differentiated or not differentiated.

Phase region: A phase region as meant herein is a region comprised on a ta-siRNA molecule being homologous to a target region and being released as 21 to 24 bp small dsRNA molecule upon processing of said ta-siRNA molecule in a plant cell. Target regions of such small dsRNA molecules derived from ta-siRNA molecules are for example the coding region of a target gene, the transcribed region of a non coding gene or the promoter of a target gene. Processing of ta-siRNAs and the prediction of phase regions are for example described in Allen et al (2005).

Plant: The terms “plant” or “plant organism” refer to any eukaryotic organism, which is capable of photosynthesis, and the cells, tissues, parts or propagation material (such as seeds or fruits) derived therefrom. Encompassed within the scope of the invention are all genera and species of higher and lower plants of the Plant Kingdom as well as algae. Annual, perennial, monocotyledonous and dicotyledonous plants and gymnosperms are preferred. A “plant” refers to any plant or part of a plant at any stage of development. Mature plants refer to plants at any developmental stage beyond the seedling stage. Encompassed are mature plant, seed, shoots and seedlings, and parts, propagation material (for example tubers, seeds or fruits) and cultures (for example cell cultures or callus cultures,) derived therefrom. Seedling refers to a young, immature plant at an early developmental stage. Therein are also included cuttings, cell or tissue cultures and seeds. As used in conjunction with the present invention, the term “plant tissue” includes, but is not limited to, whole plants, plant cells, plant organs, plant seeds, protoplasts, callus, cell cultures, and any groups of plant cells organized into structural and/or functional units. Preferably, the term “plant” as used herein refers to a plurality of plant cells, which are largely differentiated into a structure that is present at any stage of a plant's development. Such structures include one or more plant organs including, but are not limited to, fruit, shoot, stem, leaf, flower petal, etc. More preferably, the term “plant” includes whole plants, shoot vegetative organs/structures (e.g. leaves, stems and tubers), roots, flowers and floral organs/structures (e.g. bracts, sepals, petals, stamens, carpels, anthers and ovules), seeds (including embryo, endosperm, and seed coat) and fruits (the mature ovary), plant tissues (e.g. vascular tissue, ground tissue, and the like) and cells (e.g. guard cells, egg cells, trichomes and the like), and progeny of same. The class of plants that can be used in the method of the invention is generally as broad as the class of higher and lower plants amenable to transformation techniques, including angiosperms (monocotyledonous and dicotyledonous plants), gymnosperms, ferns, and multicellular algae. Included within the scope of the invention are all genera and species of higher and lower plants of the plant kingdom. Included are furthermore the mature plants, seed, shoots and seedlings, and parts, propagation material (for example seeds and fruit) and cultures, for example cell cultures, derived therefrom. Preferred are plants and plant materials of the following plant families: Amaranthaceae, Brassicaceae, Carophyllaceae, Chenopodiaceae, Compositae, Cucurbitaceae, Labiatae, Leguminosae, Papilionoideae, Liliaceae, Linaceae, Malvaceae, Rosaceae, Saxifragaceae, Scrophulariaceae, Solanaceae, Tetragoniaceae. Annual, perennial, monocotyledonous and dicotyledonous plants are preferred host organisms for the generation of transgenic plants. The use of the method according to the invention is furthermore advantageous in all ornamental plants, forestry, fruit, or ornamental trees, flowers, cut flowers, shrubs or turf. Said plant may include—but shall not be limited to—bryophytes such as, for example, Hepaticae (hepaticas) and Musci (mosses); pteridophytes such as ferns, horsetail and clubmosses; gymnosperms such as conifers, cycads, ginkgo and Gnetaeae; algae such as Chlorophyceae, Phaeophpyceae, Rhodophyceae, Myxophyceae, Xanthophyceae, Bacillariophyceae (diatoms) and Euglenophyceae. Plants for the purposes of the invention may comprise the families of the Rosaceae such as rose, Ericaceae such as rhododendrons and azaleas, Euphorbiaceae such as poinsettias and croton, Caryophyllaceae such as pinks, Solanaceae such as petunias, Gesneriaceae such as African violet, Balsaminaceae such as touch-me-not, Orchidaceae such as orchids, Iridaceae such as gladioli, iris, freesia and crocus, Compositae such as marigold, Geraniaceae such as geraniums, Liliaceae such as Drachaena, Moraceae such as ficus, Araceae such as philodendron and many others. The transgenic plants according to the invention are furthermore selected in particular from among dicotyledonous crop plants such as, for example, from the families of the Leguminosae such as pea, alfalfa and soybean; the family of the Umbelliferae, particularly the genus Daucus (very particularly the species carota (carrot)) and Apium (very particularly the species graveolens var. dulce (celery)) and many others; the family of the Solanaceae, particularly the genus Lycopersicon, very particularly the species esculentum (tomato) and the genus Solanum, very particularly the species tuberosum (potato) and melongena (aubergine), tobacco and many others; and the genus Capsicum, very particularly the species annum (pepper) and many others; the family of the Leguminosae, particularly the genus Glycine, very particularly the species max (soybean) and many others; and the family of the Cruciferae, particularly the genus Brassica, very particularly the species napus (oilseed rape), campestris (beet), oleracea cv Tastie (cabbage), oleracea cv Snowball Y (cauliflower) and oleracea cv Emperor (broccoli); and the genus Arabidopsis, very particularly the species thaliana and many others; the family of the Compositae, particularly the genus Lactuca, very particularly the species sativa (lettuce) and many others. The transgenic plants according to the invention are selected in particular among monocotyledonous crop plants, such as, for example, cereals such as wheat, barley, sorghum and millet, rye, triticale, maize, rice or oats, and sugarcane. Further preferred are trees such as apple, pear, quince, plum, cherry, peach, nectarine, apricot, papaya, mango, and other woody species including coniferous and deciduous trees such as poplar, pine, sequoia, cedar, oak, etc. Especially preferred are Arabidopsis thaliana, Nicotiana tabacum, oilseed rape, soybean, corn (maize), wheat, cotton, potato and tagetes.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refers to a DNA sequence which when ligated to a nucleotide sequence of interest is capable of controlling the transcription of the nucleotide sequence of interest into mRNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgi-bin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 bp proximal to the transcriptional start site, preferably the at least 1000 bp, more preferably the at least 500 bp, even more preferably the at least 400 bp, the at least 300 bp, the at least 200 bp or the at least 100 bp. In a further preferred embodiment, the promoter comprises the at least 50 bp proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter means that the promoter is capable of directing transcription of an operably linked nucleic acid sequence in the absence of a stimulus (e.g., heat shock, chemicals, light, etc.) in a majority of plant tissues and cells. Typically, constitutive promoters are capable of directing expression of a transgene in substantially any cell and any tissue.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

Reference Plant: “Reference plant” is any plant that is used as a reference for a genetically modified plant, for example transgenic or mutagenized plant. A reference plant preferentially is substantially identical to, more preferential a clone of the starting plant used in the respective process for transformation or mutagenization as defined above.

Regulatory box of a regulatory region: A “regulatory box of a regulatory region” as used herein means a sequence element or a motif comprised in the sequence of a regulatory region with which regulatory proteins and/or nucleic acids interact, thereby influencing the specificity of a regulatory region. A regulatory box of a regulatory region may for example be 22 bp or less, preferably 16 bp or less, more preferably 12 bp or less, even more preferably 8 bp or less long. At least, the regulatory box of a regulatory region consists of 4 bp. For example, such regulatory boxes are listed in the transfac database http://www.biobase-international.com/pages/index.php?id=transfac.

Regulatory region: A “regulatory region” or a “regulatory element” could be any region encoded on the genome and/or on the transcript influencing expression of a gene. For example, influence could mean directing or preventing expression, regulating quantity or specificity of expression. Processes that could be influenced by a regulatory region are for example transcription, translation or transcript stability. For example “regulatory regions” are promoters, promoters, enhancers, repressors, introns, 5′ and 3′ UTRs. This list is a non exclusive list. A plant specific regulatory region is a regulatory region functional in a plant. It may be derived from a plant but also from plant pathogens or it might be a synthetic regulatory region designed by man.

A “sector targeted by a sncRNA”, including sncaRNA or a “sector” means a section or part of the promoter which interacts with a sncRNA thereby regulating the expression conferred by said promoter such as increase or decrease of expression. Said interaction may be a direct interaction of the sncRNA and the promoter for example base-pairing between homologous regions of the sncRNA and the promoter. The interaction can also be the adsorption or attachment of the sncRNA to the promoter without base-pairing between the two molecules. It can in addition mean an indirect interaction, for example that the sncRNA interacts with one or more protein that then interact with the promoter.

A “sector targeted by a sncaRNA” as used herein means a nucleic acid sequence being part of a promoter the sncaRNA is interacting with. Such sector may be any region within a plant specific promoter, it may comprise completely or a part of a regulatory box of the promoter or the transcription start site of the promoter. The sector is homologous, for example 70% or more homologous, preferably 80% or more homologous, more preferably 90% or more homologous, most preferably 100% homologous to a sncaRNA, which confers upon interaction with, for example binding of a sncaRNA, an increase of the gene regulated by said promoter.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

Short hairpinRNA: A “short hairpin RNA” as used herein means a partially doublestranded RNA molecule of between about 16 to about 26 bp, for example 16 to 26 bp comprising a hairpin structure. These short hairpinRNAs are derived from the expression of recombinant constructs comprising in 5″3″ direction 16 to 26 bp followed by a short linker of about 5-50 bp followed by 16 to 26 bp being at least partially complementary to the first 16 to 26 bp followed by a 3′ untranscribed region. This construct is operably linked to the promoter of a Pol III RNA gene promoter, for example a plant specific Pol III RNA gene promoter. Upon expression of this construct the respective complementary 16 to 26 bp form a doublestranded structure whereby the linker forms a hairpin. Such constructs are for example described in Lu et al. (2004). The person skilled in the art is aware of possible variations in designing such constructs.

Significant Increase or Decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Small nucleic acid molecules: “small nucleic acid molecules” are understood as molecules consisting of nucleic acids or derivatives thereof such as RNA or DNA. They may be double-stranded or single-stranded and are between about 15 and about 30 bp, for example between 15 and 30 bp, more preferred between about 19 and about 26 bp, for example between 19 and 26 bp, even more preferred between about 20 and about 25 bp for example between 20 and 25 bp. In a especially preferred embodiment the oligonucleotides are between about 21 and about 24 bp, for example between 21 and 24 bp. In a most preferred embodiment, the small nucleic acid molecules are about 21 bp and about 24 bp, for example 21 bp and 24 bp.

Small non-coding RNA: “small non-coding RNA” or “sncRNA” as used in this document means RNAs derived from a plant or part thereof that are not coding for a protein or peptide and have a biological function as RNA molecule as such. They are for example involved in regulation of gene expression such as transcription, translation, processing of pre-mRNA and mRNA and/or RNA decay. A large number of different “sncRNAs” have been identified, differing in origin and function. “SncRNAs” are for example ta-siRNAs, shRNAs, siRNAs, microRNAs, snRNAs, nat-siRNA and/or snoRNAs. They may be double-stranded or single-stranded and are between about 10 and about 80 bp, for example between 10 and 80 bp, between about 10 and about 50 bp, for example between 10 and 50 bp, between 15 and about 30 bp, for example between 15 and 30 bp, more preferred between about 19 and about 26 bp, for example between 19 and 26 bp, even more preferred between about 20 and about 25 bp for example between 20 and 25 bp. In a especially preferred embodiment the oligonucleotides are between about 21 and about 24 bp, for example between 21 and 24 bp. In a most preferred embodiment, the sncRNAs are about 21 bp and about 24 bp, for example 21 bp and 24 bp.

Small non-coding activating RNA: “small non-coding activating RNA” or “sncaRNA” as used in this document are a subset of the sncRNAs. They are involved in regulation of gene expression. Upon interaction with promoters they lead to increased expression derived from these promoters.

Stabilize: To “stabilize” the expression of a nucleotide sequence in a plant cell means that the level of expression of the nucleotide sequence after applying a method of the present invention is approximately the same in cells from the same tissue in different plants from the same generation or throughout multiple generations when the plants are grown under the same or comparable conditions.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary” to a reference nucleotide sequence hybridizes to the reference nucleotide sequence under low stringency conditions, preferably medium stringency conditions, most preferably high stringency conditions (as defined above).

Substantially identical: In its broadest sense, the term “substantially identical”, when used herein with respect to a nucleotide sequence, means a nucleotide sequence corresponding to a reference or target nucleotide sequence, wherein the percentage of identity between the substantially identical nucleotide sequence and the reference or target nucleotide sequence is desirably at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially identical” to a reference nucleotide sequence hybridizes to the exact complementary sequence of the reference nucleotide sequence (i.e. its corresponding strand in a double-stranded molecule) under low stringency conditions, preferably medium stringency conditions, most preferably high stringency conditions (as defined above). Homologes of a specific nucleotide sequence include nucleotide sequences that encode an amino acid sequence that is at least 24% identical, more preferably at least 35% identical, yet more preferably at least 50% identical, yet more preferably at least 65% identical to the reference amino acid sequence, as measured using the parameters described above, wherein the amino acid sequence encoded by the homolog has the same biological activity as the protein encoded by the specific nucleotide. The term “substantially identical”, when used herein with respect to a polypeptide, means a protein corresponding to a reference polypeptide, wherein the polypeptide has substantially the same structure and function as the reference protein, e.g. where only changes in amino acids sequence not affecting the polypeptide function occur. When used for a polypeptide or an amino acid sequence the percentage of identity between the substantially similar and the reference polypeptide or amino acid sequence desirably is at least 24%, more desirably at least 30%, more desirably at least 45%, preferably at least 60%, more preferably at least 75%, still more preferably at least 90%, yet still more preferably at least 95%, yet still more preferably at least 99%, using default GAP analysis parameters as described above. Homologes are amino acid sequences that are at least 24% identical, more preferably at least 35% identical, yet more preferably at least 50% identical, yet more preferably at least 65% identical to the reference polypeptide or amino acid sequence, as measured using the parameters described above, wherein the amino acid sequence encoded by the homolog has the same biological activity as the reference polypeptide. The term “substantially identical”, when used herein with respect to a plant means in its broadest sense two plants of the same genus. When used with respect to a transgenic plant and a reference plant, substantially identical means that the genomic sequence of the reference plant is substantially identical to the transgenic plant with the exception of the recombinant construct the transgenic plant is bearing.

The terms “target”, “target gene” and “target nucleotide sequence” are used equivalently. As used herein, a target gene can be any gene of interest present in a plant. A target gene may be endogenous or introduced. For example, a target gene is a gene of known function or is a gene whose function is unknown, but whose total or partial nucleotide sequence is known. A target gene is a native gene of the plant cell or is a heterologous gene which has previously been introduced into the plant cell or a parent cell of said plant cell, for example by genetic transformation. A heterologous target gene is stably integrated in the genome of the plant cell or is present in the plant cell as an extrachromosomal molecule, e.g. as an autonomously replicating extrachromosomal molecule. A target gene may include polynucleotides comprising a region that encodes a polypeptide or polynucleotide region that regulates replication, transcription, translation, or other process important in expression of a target protein; or a polynucleotide comprising a region that encodes the target polypeptide and a region that regulates expression of the target polypeptide; or non-coding regions such as the 5′ or 3′ UTR or introns. A target gene may refer to, for example, an RNA molecule produced by transcription of a gene of interest. The target gene may also be a heterologous gene expressed in a recombinant cell or a genetically altered plant. In a preferred embodiment, target genes are genes improving agronomical important traits such as for example yield and yield stability, stress resistance comprising both biotic and abiotic stresses such as fungal or drought resistance. Other agronomic important traits are for example the content of vitamins, amino acids, PUFAs or other metabolites of interest.

Tissue: The term “tissue” with respect to a plant means arrangement of multiple cells including differentiated and undifferentiated tissues of the organism. Tissues may constitute part of an organ (e.g., the epidermis of a plant leaf) but may also constitute tumor tissues (e.g., callus tissue) and various types of cells in culture (e.g., single cells, protoplasts, embryos, calli, etc.). The tissue may be in vivo (e.g., in planta), in organ culture, tissue culture, or cell culture.

Transformation: The term “transformation” as used herein refers to the introduction of genetic material (e.g., a transgene or heterologous nucleic acid molecules) into a plant cell, plant tissue or plant. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzyme-linked immunosorbent assay (ELISA), which detects the presence of a polypeptide encoded by one or more of the transgenes. Alternatively, transient transformation may be detected by detecting the activity of the protein (e.g., R-glucuronidase) encoded by the transgene (e.g., the uid A gene). The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences, which are capable of binding to one or more of the transgenes. Alternatively, stable transformation of a cell may also be detected by the polymerase chain reaction of genomic DNA of the cell to amplify transgene sequences. The term “stable transformant” refers to a cell, which has stably integrated one or more transgenes into the genomic DNA. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression, which may exhibit variable properties with respect to meiotic stability. Transformed cells, tissues, or plants are understood to encompass not only the end product of a transformation process, but also transgenic progeny thereof.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to a plant cell, plant tissue or plant means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### EXAMPLES

### Example 1

### Arabidopsis Protoplast Transformation and Hormone-Inducible Promoter-Reporter Assay

Materials and Methods

Plant Material: Four weeks old Arabidopsis plants of ecotype col-0 were used for the experiments.

Plasmid Constructs:

Experiments were conducted using 2 different promoter::reporter constructs. GH3-LUC induced by IAA and RD29A-LUC (Kovtun et al., 2000, Por. Natl. acad. Sci. USA 97:2940-2945) induced by ABA were obtained from the Arabidopsis Biological Resource Center (www.biosci.ohio-state.edu/˜plantbio/Facilities/abrc/abrccontactus.htm).

Protoplast Isolation:

Well expanded healthy leaves were used for protoplast isolation. Protoplasts were isolated as described by Yoo et al., (2007, Nature protocols 2(7):1565-1572) with some modifications. About 10-20 leaves were digested in 10 ml of enzyme solution which contained 1.5% cellulose and 0.3% macerozyme Leaves were cut into 0.5-1 mm leaf strips and dipped in the enzyme solution and vacuum infiltrated for 3 minutes. At the end of 3 minutes the vacuum was disconnected quickly to force the enzyme solution into the leaf slices. The procedure was repeated 3 times. Leaves were left in the enzyme solution overnight.

Protoplast Transformation:

1×10⁴ protoplasts were transformed with 10 μg plasmid DNA using PEG (poly ethylene glycol). The transformed protoplasts were incubated for 16 h in the dark with 1 μM IAA for the protoplasts transformed with GH3-LUC and 100 μM ABA for the protoplasts transformed with RD29A-LUC. Controls were mock transformed protoplasts and protoplasts that were transformed with their relative plasmids but not treated with either IAA or ABA.

For experiments with siRNA, 1×10⁴ protoplasts were co-transformed with 10 μg reporter plasmid and 5 μg of siRNA.

Luciferase Assay

Luciferase assay was done using the luciferase Assay System (Promega) according to the manufacturer's instructions. Protoplasts were pelleted, 100 μl of cell lysis buffer was added to the pellet, vortexed and centrifuged. To 20 μl of supernatant 100 μl of assay buffer was added and luminescence was read using a luminometer (Lmax). The results shown are shown as means of relative LUC activities from triplicate samples along with error bars. All experiments were repeated 3 times with similar results. In the presence of IAA and ABA, we were able to induce luciferase expression with the addition of 1 μM IAA or 100 μM ABA as previously reported by Hwang & Sheen (2001)

### Example 2

### Design siRNAs to Target Hormone-Inducible Promoters

To test activation of gene expression by small RNAs we designed a number of siRNAs whose sequence corresponds to fragments of the ABA and IAA promoter sequences. Twenty-one nucleotide synthetic duplex RNAs were designed where there was an overlap of 19 nucleotides with two nucleotide 3′ overhangs on both the sense and antisense strands. siRNAs were designed to correspond to promoter sequences from 100 nucleotides upstream of the TATA box to the 3′ end of the promoter.

ABA Inducible Promoter

ABA promoter (SEQ ID NO: 1) siRNAs were designed to cover a 216 base pair region that spans from 100 nucleotides upstream of the TATA box to the end of the promoter (positions 141 to 356 of SEQ ID NO: 1). Twenty-one nucleotide siRNAs were designed to start at position 141 of SEQ ID NO: 1 and walk along the remaining length of the promoter, advancing 5 nucleotides at a time, in the 5′ to 3′ direction. A total of 40 siRNAs were designed to cover the region from position 141 to 356 of SEQ ID NO: 1.

For example, the first siRNA designed for the ABA promoter, named A-1, contains a sense strand that corresponds to positions 141 to 161 of SEQ ID NO: 1. The antisense strand of siRNA A-1 is the reverse complement to positions 139 to 159 of SEQ ID NO: 1. The sense and anti-sense siRNAs are annealed to make siRNA duplex with 3′ 2 nt overhangs. For example, A-1 siRNA duplex contains sense (SEQ ID NO: 22) and anti-sense (SEQ ID NO: 23) of A-1 small activating RNAs. The second siRNA, named A-2, designed for the ABA promoter contains a sense strand that corresponds to position 146 to 166 of SEQ ID NO: 1. The antisense strand of siRNA A-2 is the reverse complement to positions 144 to 164 of SEQ ID NO: 1. siRNAs were designed to cover the remaining ABA promoter sequence using the same design as siRNA A-1 and A-2.

IAA Inducible Promoter

The IAA promoter (SEQ ID NO: 2) contains two potential TATA boxes. IAA promoter (SEQ ID NO: 2) siRNAs were designed to cover a 761 base pair region that spans from 100 nucleotides upstream of the first TATA box to the end of the promoter (positions 2753 to 3513 of SEQ ID NO: 2). Twenty-one nucleotide siRNAs were designed to start at position 2753 of SEQ ID NO: 2 and walk along the remaining length of the promoter, advancing 5 nucleotides at a time, in the 5′ to 3′ direction. A total of 149 siRNAs were designed to cover the region from position 2753 to 3513 of SEQ ID NO: 2.

For example, the first siRNA designed for the IAA promoter, named I-1, contains a sense strand that corresponds to positions 2753 to 2773 of SEQ ID NO: 2. The antisense strand of siRNA I-1 is the reverse complement to positions 2751 to 2771 of SEQ ID NO: 2. The sense and anti-sense siRNAs are annealed to make siRNA duplex with 3′ 2 nt overhangs. For example, I-24 siRNA duplex contains sense (SEQ ID NO: 6) and anti-sense (SEQ ID NO: 7) of I-24 small activating RNAs. The second siRNA, named I-2, designed for the IAA promoter contains a sense strand that corresponds to position 2758 to 2778 of SEQ ID NO: 2. The antisense strand of siRNA I-2 is the reverse complement to positions 2756 to 2776 of SEQ ID NO: 2. siRNAs were designed to cover the remaining IAA promoter sequence using the same design as siRNA I-1 and I-2.

ACC Inducible Promoter

ACC inducible promoter (SEQ ID NO: 3) siRNAs were designed to cover the complete promoter region (positions 1 to 146 of SEQ ID NO: 3). Twenty-one nucleotide siRNAs were designed to start at position 1 of SEQ ID NO: 3 and walk along the remaining length of the promoter, advancing five nucleotides at a time, in the 5′ to 3′ direction. A total of 26 siRNAs were designed to cover this region.

Zeatin Inducible Promoter

ABA inducible promoter (SEQ ID NO: 4) siRNAs were designed to cover a 411 base pair region that spans from 200 nucleotides upstream of the TATA box to the end of the promoter (positions 1987 to 2397 of SEQ ID NO: 4). Twenty-one nucleotide siRNAs were designed to start at position 1987 of SEQ ID NO: 4 and walk along the remaining length of the promoter, advancing five nucleotides at a time, in the 5′ to 3′ direction. A total of 79 siRNAs were designed to cover the region from position 1987 to 2397 of SEQ ID NO: 4.

### Example 3

### Test Activation of Hormone-Inducible Promoters by siRNAs in Arabidopsis Protoplast System

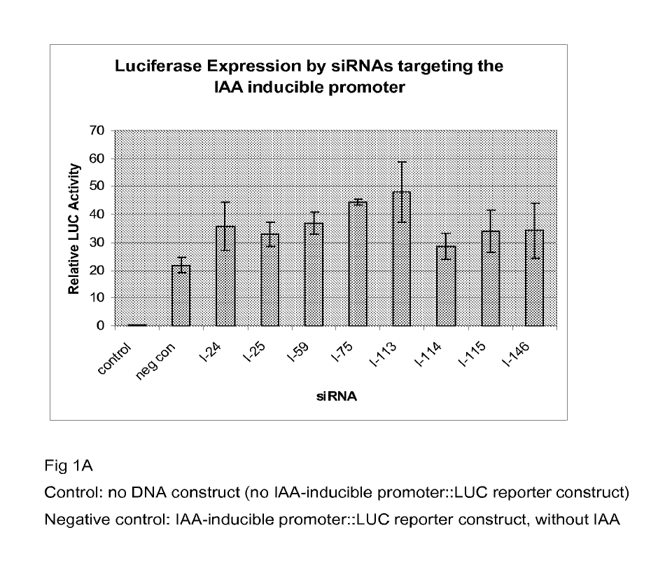

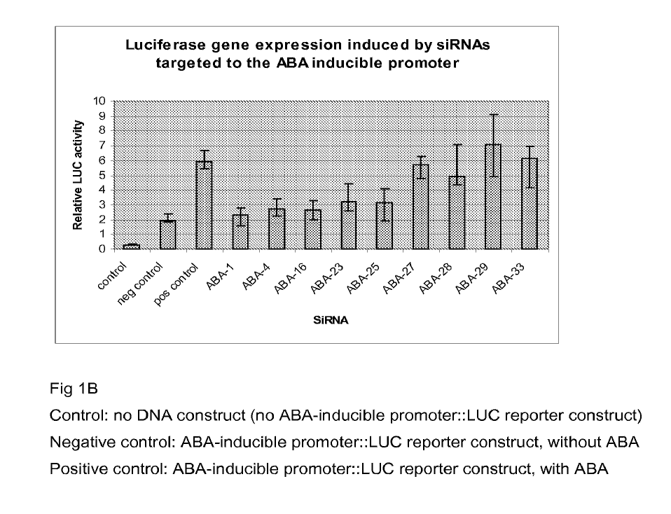

Out of the 149 siRNAs targeted for the GH3-LUC promoter, 8 of them activated luciferase gene expression in the absence of IAA (FIG. 1A). For the RD29A-LUC promoter 9 of the 40 siRNAs tested showed elevated luciferase expression in the absence of ABA (FIG. 1B).

We characterized the GH3-LUC and RD29A-LUC promoter using Genomatix for transcription factor binding sites. Interestingly we found that our hits were around the TATA box region or regulatory elements including transcriptional repressor BELLRINGER, promoters of different sugar responsive genes, Ellicitor response element, ABA inducible transcriptional activator, Rice Transcription activator-1, TCP class II transcription factor, auxin response element and CA rich element.

Negative control: hormone-inducible promoter::LUC reporter, no hormone

Positive control: hormone-inducible promoter::LUC reporter with hormone

TABLE 1
siRNAs to the GH3-LUC promoter that activated luciferase espression
(with SEQ ID NO) and the siRNAs sorrounding them

| | SEQ | | SEQ | | nucleotide | | |
|---|---|---|---|---|---|---|---|
| siRNA | ID | | ID | | positions of | | |
| name | NO | Sense sequence | NO | Anti-sense sequence | SEQ ID NO: 2 | | |
| I-21 | | uuauuuuauauacagaauucc | | aauucuguauauaaaauaaag | 2853 | 2873 | TATA BOX |
| I-22 | | uuauauacagaauuccggauu | | uccggaauucuguauauaaaa | 2858 | 2878 | 2852-66 |
| I-23 | | uacagaauuccggauuaugag | | cauaauccggaauucuguaua | 2863 | 2883 | |
| I-24 | 6 | aauuccggauuaugagagaaa | 7 | ucucucauaauccggaauucu | 2868 | 2888 | |
| I-25 | 8 | cggauuaugagagaaaaaaac | 9 | uuuuuucucucauaauccgga | 2873 | 2893 | |
| I-59 | 10 | accaagucuucuuaauucuga | 11 | agaauuaagaagacuugguua | 3043 | 3063 | |
| I-75 | 12 | uuuaguauugaguauugaccg | 13 | gucaauacucaauacuaaaag | 3123 | 3143 | 3132-3148- |
| I-76 | | uauugaguauugaccgucgcu | | cgacggucaauacucaauacu | 3128 | 3148 | Ellicitor |
| | | | | | | | response |
| | | | | | | | element |
| I-112 | | caaagauuacgugaccgcggu | | cgcggucacguaaucuuuggc | 3308 | 3328 | 3309-3325- |
| I-113 | 14 | auuacgugaccgcggucccuc | 15 | gggaccgcggucacguaaucu | 3313 | 3333 | ABA inducible |
| | | | | | | | transcriptional |
| | | | | | | | activator |
| I-114 | 16 | gugaccgcggucccucuuguc | 17 | caagagggaccgcggucacgu | 3318 | 3338 | 3310-3326 |
| | | | | | | | Rice |
| | | | | | | | Transcription |
| | | | | | | | activator-1 |
| I-115 | 18 | cgcggucccucuuguccccug | 19 | ggggacaagagggaccgcggu | 3323 | 3343 | 3323-3335 |
| | | | | | | | TCPclass II |
| | | | | | | | transcription |
| | | | | | | | factor |
| I-116 | | ucccucuuguccccugucucg | | agacaggggacaagagggacc | 3328 | 3348 | 3332-3344 |
| | | | | | | | Auxin |
| | | | | | | | response |
| | | | | | | | element |
| I-146 | 20 | acaaagucuaauauuaucacu | 21 | ugauaauauuagacuuugugu | 3478 | 3498 | 3468-3486- |
| | | | | | | | CA rich |
| | | | | | | | element |

TABLE 2
siRNAs to the RD29A promoter that activated luciferase expression
(with SEQ ID NO) and the siRNAs surrounding them

| | SEQ | | SEQ | | nucleotide | | |
|---|---|---|---|---|---|---|---|
| siRNA | ID | | ID | | position of | | |
| name | NO | Sense sequence | NO | Anti-sense sequence | SEQ ID NO: 1 | | |
| A-1 | 22 | aagaucaagccgacac | 23 | ucugugucggcuugaucuu | 141 | 161 | |
| | | agaca | | uu | | | |
| A-4 | 24 | cagacacgcguagaga | 25 | ugcucucuacgcgugucug | 156 | 176 | |
| | | gcaaa | | ug | | | |
| A-16 | 26 | cgugucccuuuaucuc | 27 | agagagauaaagggacacg | 216 | 236 | |
| | | ucuca | | ua | | | |
| A-21 | | cucuauaaacuuagu | | ucucacuaaguuuauagag | 241 | 261 | |
| | | gagacc | | ag | | | |
| A-22 | | uaaacuuagugagac | | gagggucucacuaaguuua | 246 | 266 | 247-257- |
| | | ccuccu | | ua | | | Transcriptional |
| A-23 | 28 | uuagugagacccuccu | 29 | cagaggagggucucacuaa | 251 | 271 | repressor |
| | | cuguu | | gu | | | BELLRINGER |
| A-25 | 30 | ccuccucuguuuuacu | 31 | gugaguaaaacagaggagg | 261 | 281 | |
| | | cacaa | | gu | | | |
| A-27 | 32 | uuuacucacaaauau | 33 | uugcauauuugugaguaaa | 271 | 291 | |
| | | gcaaac | | ac | | | |
| A-28 | 34 | ucacaaauaugcaaac | 35 | cuaguuugcauauuuguga | | | |
| | | uagaa | | gu | 276 | 296 | |
| A-29 | 36 | aauaugcaaacuaga | 37 | guuuucuaguuugcauauu | | | 297-315- |
| | | aaacaa | | ug | 281 | 301 | promoters of |
| A-33 | 38 | aucaucaggaauaaa | 39 | acccuuuauuccugaugau | 301 | 321 | different sugar |
| | | ggguuu | | ug | | | responsive |
| | | | | | | | genes |

### Example 4

### In Silico Identification of Candidate Genes Targeted by Endogenous miRNAs in the Regulatory Regions

Over 100 known Arabidopsis microRNAs were extracted from Mirbase (http://microrna.sanger.ac.uk) and searched against a TAIR database (www.arabidopsis.org/) consisting of up to 3 kilobase regions upstream of every gene in Arabidopsis. We searched these putative promoter regions that may comprise 5″untranslated regions in both frames with the known Arabidopsis microRNAs as queries using ungapped BLAST with a reduced word size of 7 as a pre-filter and then re-aligned the regions using the Smith-Waterman algorithm. We then required the following conditions for an alignment to be called a potential target. All of these requirements are indexed off of the 5′ most base of the known microRNA.

First, no more than 4 total mismatches.

Second, no mismatches at base10 or 11.

Third, no more than one mismatch is allowed between bases 2 and 9.

Fourth, if there is a mismatch between by 2 and 9, no more than 2 other mismatches in the alignment.

Fifth, no more than 2 consecutive mismatches from base 12 through 21.

All alignments which met the above conditions were considered a promoter target for the microRNA.

We further limited miRNA hits within the 2 Kb upstream putative promoter regions, and found that the sense strand of the promoter of 853 genes and the antisense strand of the promoter of 651 genes are targeted by 107 known miRNAs. We then picked one miRNA/family and thus identified 214 miRNAs that target the sense strand and 171 miRNAs that target the antisense strand.

### Example 5

### Test Activation and Up-Regulation of Genes Whose Promoter were Targeted by Endogenous miRNAs

We used PCR to isolate precursors of miRNAs listed in Table 1 from Arabidopsis. The precursors are 800-1000 bp in length. The PCR product was first TA cloned into Gateway 5′entry vector ENTR 5′-TOPO (Invitrogen #K591-20). Plant binary expression vectors were constructed through multi-site Gateway cloning by combining three entry vectors containing promoter, gene of interest and terminator and one destination vector in a LR reaction (Invitrogen #K591-10). Thus, expression of each miRNA precursor is under the control of parsley ubiquitin promoter and terminator from nopaline synthase. The final binary vectors were confirmed by sequencing. Arabidopsis plants col-0 were transformed with the constructs using the flower dip method (Clough and Bent, 1998, Plant J 16:735-43) to generate transgenic lines.

We used qRT-PCR to determine activation and up-regulation of genes whose promoter targeted by a miRNA in transgenic Arabidopsis plants that over-express the miRNA. Seeds were germinated on MS medium supplemented with 10 mg/l Phosphinothricin (PPT). RNA was extracted from 3 weeks old plants using the RNeasy plant Mini kit (Qiagen) from three independent events. Five plants per event were pooled together. qRT-PCR was done using sybr Green. A total of 43 genes were tested. The gene(s) is predicted to be targeted by the same miRNA in the coding region was included in qRT-PCR. Arabidopsis tubulin or actin gene was used an endogenous control to normalize relative expression. The genes that were up-regulated were further confirmed by TaqMan.

Out of the 214 and 172 miRNAs targeting the sense and the antisense strands of putative promoters respectively, we tested 12 miRNAs to see for any up-regulation of the genes of these promoters. Using the qRT-PCR method, we identified miR159b that upregulated the gene (AT3G50830) and miR398a that upregulated the gene (AT3G15500) by targeting their promoters, respectively.

### Example 6

### Further Analysis of siRNAs Targeting Hormone-Inducible Promoter

Mutated siRNAs

From the initial experiments nine siRNAs corresponding to regions of the ABA inducible promoter were found to activate gene expression. Eight siRNAs corresponding to regions of the IAA inducible promoter were found to activate gene expression. Make mutations in the siRNAs discovered in the initial ABA and IAA inducible promoter experiments to have the ability to activate gene expression. Specific nucleotide or nucleotides will be changed in the siRNA duplexes to study the effect specific positions have on gene activation.

### Positions 9, 10, and 11

Design siRNAs to test the necessity of positions nine, ten, and eleven having a perfect match to their promoter target regions are for RNA induced gene activation. Mutate positions nine, ten, and eleven of the sense strand of the functional siRNAs A-23, A-25, A-27, A-28, A-29, A-33, and I-24, I-25, I-113, I-114, I-115. Make the corresponding mutations in the anti-sense strand of the duplex siRNAs. Maintain the same G/C content as the functional siRNAs when making the mutations. The mutated nucleotides are represented as upper case letters in the table below. SiRNAs with mutations at positions nine, ten, and eleven produced comparable results to the original siRNAs that are perfectly homologous to the promoter target regions. Mismatches at positions nine, ten, and eleven between the siRNA and the targeted promoter region does not significantly affect RNAa activity.

TABLE 3
siRNAs mutated at positions 9, 10 and 11

| Original | Mutated | | |
|---|---|---|---|
| siRNA | siRNA | Sense sequence | Anti-sense sequence |
| A-23 | A-67 | uuagugagUGGcuccucuguu | cagaggagCCAcucacuaagu |
| A-25 | A-68 | ccuccucuCAAuuacucacaa | gugaguaaUUGagaggagggu |
| A-27 | A-69 | uuuacucaGUUauaugcaaac | uugcauauAACugaguaaaac |
| A-28 | A-70 | ucacaaauUACcaaacuagaa | cuaguuugGUAauuugugagu |
| A-29 | A-71 | aauaugcaUUGuagaaaacaa | guuuucuaCAAugcauauuug |
| A-33 | A-72 | aucaucagCUUuaaaggguuu | acccuuuaAAGcugaugauug |
| I-24 | I-171 | aauuccggUAAaugagagaaa | ucucucauUUAccggaauucu |
| I-25 | I-172 | cggauuauCUCagaaaaaaac | uuuuuucuGAGauaauccgga |
| I-113 | I-173 | auuacgugUGGgcggucccuc | gggaccgcCCAcacguaaucu |
| I-114 | I-174 | gugaccgcCCAcccucuuguc | caagagggUGGgcggucacgu |
| I-115 | I-175 | cgcgguccGAGuuguccccug | ggggacaaCUCggaccgcggu |

Positions 4, 5, and 6

Design siRNAs to test if mismatches in the 5′ end of siRNAs and the promoter target regions have an affect on gene activation. Mutate positions four, five, and six of the sense and anti-sense strands individually of functional siRNA duplexes. Make the corresponding mutations in the anti-sense strand of the duplex siRNAs. Mutate previously identified functional siRNAs A-27, A-28, I-113, and I-114. Maintain the same G/C content as the functional siRNAs when making the mutations. The mutated nucleotides are represented as upper case letters in the table below. SiRNAs with mutations at positions four, five, and six lost RNAa activity. Mismatches at positions four, five, and six between the siRNA and the targeted promoter region significantly reduced RNAa activity when compared to the original, previously identified functional siRNAs.

TABLE 4
siRNAs mutated at positions 4, 5 and 6

| Original | Mutated | Sense | Anti-sense |
|---|---|---|---|
| siRNA | siRNA | sequence | sequence |
| A-27 | A-87 | uuuUGAcacaaauaug | uugcauauuugugUCA |
| | | caaac | aaaac |
| A-27 | A-88 | uuuacucacaaauUAC | uugGUAauuugugagu |
| | | caaac | aaaac |
| A-28 | A-89 | ucaGUUauaugcaaac | cuaguuugcauauAAC |
| | | uagaa | ugagu |
| A-28 | A-90 | ucacaaauaugcaUUG | cuaCAAugcauauuug |
| | | uagaa | ugagu |
| I-113 | I-190 | auuUGCugaccgcggu | gggaccgcggucaGCA |
| | | cccuc | aaucu |
| I-113 | I-191 | auuacgugaccgcCCA | gggUGGgcggucacgu |
| | | cccuc | aaucu |
| I-114 | I-192 | gugUGGgcggucccuc | caagagggaccgcCCA |
| | | uuguc | cacgu |
| I-114 | I-193 | gugaccgcgguccGAG | caaCUCggaccgcggu |
| | | uuguc | cacgu |

### Positions 16, 17, and 18

Design siRNAs to test if mismatches in the 3′ end of siRNAs and the promoter target regions have an affect on gene activation. Mutate positions 16, 17, and 18 of the sense and anti-sense strands individually of functional siRNA duplexes. Make the corresponding mutations in the anti-sense strand of the duplex siRNAs. Mutate previously identified functional siRNAs A-27, A-28, I-113, and I-114. Maintain the same G/C content as the functional siRNAs when making the mutations. The mutated nucleotides are represented as upper case letters in the table below. SiRNAs with mutations at positions sixteen, seventeen, and eighteen lost RNAa activity. Mismatches at positions sixteen, seventeen, and eighteen between the siRNA and the targeted promoter region significantly reduced RNAa activity when compared to the original, previously identified functional siRNAs.

TABLE 5
siRNAs mutated at positions 16, 17 and 18

| Original | Mutated | Sense | Anti-sense |
|---|---|---|---|
| siRNA | siRNA | sequence | sequence |
| A-27 | A-91 | uuuacucacaaauauC | uACGauauuugugagu |
| | | GUaac | aaaac |
| A-27 | A-92 | uAAUcucacaaauaug | uugcauauuugugagA |
| | | caaac | UUaac |
| A-28 | A-93 | ucacaaauaugcaaaG | cAUCuuugcauauuug |
| | | AUgaa | ugagu |
| A-28 | A-94 | uGUGaaauaugcaaac | cuaguuugcauauuuC |
| | | uagaa | ACagu |
| I-113 | I-194 | auuacgugaccgcggA | gCCUccgcggucacgu |
| | | GGcuc | aaucu |
| I-113 | I-195 | aAAUcgugaccgcggu | gggaccgcggucacgA |
| | | cccuc | UUucu |
| I-114 | I-196 | gugaccgcggucccuG | cUUCagggaccgcggu |
| | | AAguc | cacgu |
| I-114 | I-197 | gACUccgcggucccuc | caagagggaccgcggA |
| | | uuguc | GUcgu |

### Position 1

Design siRNAs to test the effect the first nucleotide of a siRNA has on RNA activation. Change the first nucleotide of siRNAs previously shown to up-regulate gene expression in the initial ABA and IAA inducible promoter experiments. Select two gene activation siRNAs for each the ABA and IAA inducible promoters. Test all possible nucleotides at the first position of each strand of the siRNA duplexes. Mutate the first nucleotide of the sense and anti-sense strands individually and test the effect on RNA activation. Mutate the first position of functional siRNAs A-27, A-28, I-113 and I-114. Make the corresponding mutations in the anti-sense strand of the duplex siRNAs. The mutated nucleotides are represented as upper case letters in the table below. SiRNAs with mutations at the first nucleotide produced comparable results to the original siRNAs that are perfectly homologous to the promoter target regions.

TABLE 6
siRNAs with nucleotides changed in the first
position

| Original | Mutated | Sense | Anti-sense |
|---|---|---|---|
| siRNA | siRNA | sequence | sequence |
| A-27 | A-73 | Auuacucacaaauaug | uugcauauuugugagu |
| | | caaac | aaUac |
| A-27 | A-74 | Guuacucacaaauaug | uugcauauuugugagu |
| | | caaac | aaCac |
| A-27 | A-75 | Cuuacucacaaauaug | uugcauauuugugagu |
| | | caaac | aaGac |
| A-27 | A-76 | uuuacucacaaauaug | Augcauauuugugagu |
| | | caUac | aaaac |
| A-27 | A-77 | uuuacucacaaauaug | Gugcauauuugugagu |
| | | caCac | aaaac |
| A-27 | A-78 | uuuacucacaaauaug | Cugcauauuugugagu |
| | | caGac | aaaac |
| A-28 | A-79 | Acacaaauaugcaaac | cuaguuugcauauuug |
| | | uagaa | ugUgu |
| A-28 | A-80 | Gcacaaauaugcaaac | cuaguuugcauauuug |
| | | uagaa | ugCgu |
| A-28 | A-81 | Ccacaaauaugcaaac | cuaguuugcauauuug |
| | | uagaa | ugGgu |
| A-28 | A-82 | ucacaaauaugcaaac | Auaguuugcauauuug |
| | | uaUaa | ugagu |
| A-28 | A-83 | ucacaaauaugcaaac | Uuaguuugcauauuug |
| | | uaAaa | ugagu |
| A-28 | A-84 | ucacaaauaugcaaac | Guaguuugcauauuug |
| | | uaCaa | ugagu |
| I-113 | I-176 | Uuuacgugaccgcggu | gggaccgcggucacgu |
| | | cccuc | aaAcu |
| I-113 | I-177 | Guuacgugaccgcggu | gggaccgcggucacgu |
| | | cccuc | aaCcu |
| I-113 | I-178 | Cuuacgugaccgcggu | gggaccgcggucacgu |
| | | cccuc | aaGcu |
| I-113 | I-179 | auuacgugaccgcggu | Aggaccgcggucacgu |
| | | ccUuc | aaucu |
| I-113 | I-180 | auuacgugaccgcggu | Uggaccgcggucacgu |
| | | ccAuc | aaucu |
| I-113 | I-181 | auuacgugaccgcggu | Cggaccgcggucacgu |
| | | ccGuc | aaucu |
| I-114 | I-182 | Augaccgcggucccuc | caagagggaccgcggu |
| | | uuguc | caUgu |
| I-114 | I-183 | Uugaccgcggucccuc | caagagggaccgcggu |
| | | uuguc | caAgu |
| I-114 | I-184 | Cugaccgcggucccuc | caagagggaccgcggu |
| | | uuguc | caGgu |
| I-114 | I-185 | gugaccgcggucccuc | Aaagagggaccgcggu |
| | | uuUuc | cacgu |
| I-114 | I-186 | gugaccgcggucccuc | Uaagagggaccgcggu |
| | | uuAuc | cacgu |
| I-114 | I-187 | gugaccgcggucccuc | Gaagagggaccgcggu |
| | | uuCuc | cacgu |

### Position 20 and 21 to TT

Design siRNAs to test the effect of simultaneously changing positions 20 and 21 on the sense and anti-sense strands of the siRNA duplex has on gene activation. Mutate positions 20 and 21 on both strands of functional siRNAs A-27, A-28, I-113 and I-114. The mutated nucleotides are represented as upper case letters in the table below. SiRNAs with deoxynibocleutides TT at positions 20 and 21 produced comparable results to the original siRNAs that are perfectly homologous to the promoter target regions.

TABLE 7
siRNAs with positions 20 and 21 changed to TT

| Original | Mutated | Sense | Anit-sense |
|---|---|---|---|
| siRNA | siRNA | sequence | sequence |
| A-27 | A-85 | uuuacucacaaauaug | uugcauauuugugagu |
| | | caaTT | aaaTT |
| A-28 | A-86 | ucacaaauaugcaaac | cuaguuugcauauuug |
| | | uagTT | ugaTT |
| I-113 | I-188 | auuacgugaccgcggu | gggaccgcggucacgu |
| | | cccTT | aauTT |
| I-114 | I-189 | gugaccgcggucccuc | caagagggaccgcggu |
| | | uugTT | cacTT |

Motif Based siRNAs

Design siRNAs corresponding to specific promoter motifs in the ABA and IAA inducible promoters to determine their effects on gene activation. Design two siRNAs for each promoter motif to be targeted. The first siRNA designed to target a given motif will contain the motif sequence at the 5′ end of the appropriate sense or anti-sense strand of the duplex siRNA. The second siRNA designed to target a given motif will contain the motif sequence in the middle of the appropriate sense or anti-sense strand of the duplex siRNA. The motifs are underlined in the table below. Motif based siRNAs showed no significant ability to activate gene expression.

TABLE 8
siRNAs with motifs from the ABA inducible promoter

| | | Sense | Anit-sense |
|---|---|---|---|
| Motif | siRNA | sequence | sequence |
| Bellringer | A-95 | uacuaauaauagua | uaacuuacuauuau |
| | | aguuaca | uaguagu |
| Bellringer | A-96 | auaauaguaaguua | aaauguaacuuacu |
| | | cauuuua | auuauua |
| Zinc- | A-97 | ugacuuugacguca | uggugugacgucaa |
| finger, | | caccacg | agucauu |
| pathogen | | | |
| defence | | | |
| Zinc- | A-98 | aaaugacuuugacg | ugugacgucaaagu |
| finger, | | ucacacc | cauuuug |
| pathogen | | | |
| defence | | | |
| RITA | A-99 | acuuugacgucaca | cguggugugacguc |
| | | ccacgaa | aaaguca |
| RITA | A-100 | ugacuuugacguca | uggugugacgucaa |
| | | caccacg | agucauu |
| Zinc- | A-101 | ugacgucacaccac | uuuucgugguguga |
| finger, salt | | gaaaaca | cgucaaa |
| tolerance | | | |
| Zinc- | A-102 | cgucacaccacgaa | gucuuuucguggug |
| finger, salt | | aacagac | ugacguc |
| tolerance | | | |
| ABA | A-103 | gcuucauacguguc | aaagggacacguau |
| response | | ccuuuau | gaagcgu |
| ABA | A-104 | acgcuucauacgug | agggacacguauga |
| response | | ucccuuu | agcgucu |

TABLE 9
siRNAs with motifs from the IAA inducible promoter

| | | Sense | Anit-sense |
|---|---|---|---|
| Motif | siRNA | sequence | sequence |
| Sugar | I-198 | auguauauuauuga | aaaaaucaauaaua |
| response | | uuuuucu | uacauca |
| promoter #1 | | | |
| Sugar | I-199 | uguauauuauugau | gaaaaaucaauaau |
| response | | uuuucuu | auacauc |
| promoter #1 | | | |
| MADS-box | I-200 | uuaucaauaaauag | uacuccuauuuauu |
| | | gaguacc | gauaacu |
| Sugar | I-201 | guuuucgaaaauga | uaaaaucauuuucg |
| response | | uuuuaua | aaaacau |
| promoter #2 | | | |
| Sugar | I-202 | uuuucgaaaaugau | auaaaaucauuuuc |
| response | | uuuauaa | gaaaaca |
| promoter #2 | | | |
| Bellringer #1 | I-203 | gaauuuauuacuca | aauuuugaguaaua |
| | | aaauuaa | aauucau |
| Bellringer #1 | I-204 | gucaugaauuuauu | ugaguaauaaauuc |
| | | acucaaa | augacua |
| Bellringer #2 | I-205 | cggucaugacaaua | caauuuauugucau |
| | | aauugcc | gaccgua |
| Bellringer #2 | I-206 | augacaauaaauug | uugggcaauuuauu |
| | | cccaauc | gucauga |
| ABA inducible | I-207 | aaagauuacgugac | ccgcggucacguaa |
| TA #1 | | cgcgguc | ucuuugg |
| ABA inducible | I-208 | ccaaagauuacgug | gcggucacguaauc |
| TA #1 | | accgcgg | uuuggcu |
| Auxin | I-209 | ucuuguccccuguc | accgagacagggga |
| response | | ucggucu | caagagg |
| element | | | |
| Auxin | I-210 | ucccucuugucccc | agacaggggacaag |
| response | | ugucucg | agggacc |
| element | | | |
| ABA inducible | I-211 | uaugucgacgugga | caaauuccacgucg |
| TA #2 | | auuuggc | acauaaa |
| ABA inducible | I-212 | uuuaugucgacgug | aauuccacgucgac |
| TA #2 | | gaauuug | auaaaag |

Hot Spot Based siRNAs

From the initial ABA and IAA inducible promoter experiments specific regions of a promoter may show more ability to activate gene expression when targeted with siRNAs. Design siRNAs that walk along the promoter regions of interest, advancing two nucleotides at a time, in the 5′ to 3′ direction.

One region in the ABA inducible promoter may show greater activity when targeted with siRNAs. ABA inducible promoter (SEQ ID NO: 1) siRNAs were designed to cover a 71 base pair region that spans from positions 251 to 321 of SEQ ID NO: 1. A total of 26 siRNAs were designed to cover this region.

Two regions in the IAA inducible promoter may show greater activity when targeted with siRNAs. IAA inducible promoter (SEQ ID NO: 2) hot spot #1 is a 49 base pair region that spasm from positions 2868 to 2916 of SEQ ID NO: 2. Fifteen siRNAs were designed to cover the IAA inducible promoter hot spot #1 region. IAA inducible promoter (SEQ ID NO: 2) hot spot #2 is a 31 base pair region that spasm from positions 3313 to 3343 of SEQ ID NO: 2. Six siRNAs were designed to cover the IAA inducible promoter hot spot #2 region.

### Example 7

### Deliver Small Activating RNAs in Plant by Using a microRNA Precursor

From the initial experiments nine siRNAs corresponding to regions of the ABA inducible promoter were found to activate gene expression. Eight siRNAs corresponding to regions of the IAA inducible promoter were found to activate gene expression. These 17 siRNAs were engineered into the 272 base pair fragment Arabidopsis thaliana microRNA precursor for ath-miR164b (SEQ ID NO: 5). The wild-type microRNA sequence (positions 33-53 of SEQ ID NO: 5) was replaced with the sense strand sequence of the siRNAs discovered to activate gene expression from the initial experiment. The wild-type microRNA star sequence (positions 163-183 of SEQ ID NO: 5) was replaced with the anti-sense strand sequence of the siRNAs discovered to activate gene expression from the initial experiment.

The engineered ath-pri-miR164b containing the replaced sense and anti-sense siRNA sequences was synthesized and cloned downstream from a Parsley ubiquitin promoter. The terminator used is the 3′UTR of nopaline synthase from Agrobacterium tumefaciens T-DNA.

Luciferase gene activation was seen in the engineered siRNA constructs in at least 4 constructs from the regions of the ABA inducible promoter (SEQ ID NO:1), corresponding to siRNAs A-16 (RTP3362-1 SEQ ID NO:40), A-23 (RTP3363-1 SEQ ID NO:41), A-25 (RTP3364-1, SEQ ID NO:42) and A-27 (RTP3365-1, SEQ ID NO:43). For the constructs from the regions of the IAA inducible promoter (SEQ ID NO: 2), three of them showed activation which correspond to siRNAs I-114 (RTP3374-1, SEQ ID NO:45), I-115 (RTP3375-1, SEQ ID NO:46) and I-146 (RTP3376, SEQ ID NO: 47). The level of activation was similar to that seen using their respective synthetic siRNAs. No significant activation was observed by RTP3377-1 (SEQ ID NO:44) which produces random siRNA as a negative control.

RTP3362-1 produces small activating RNA targeting ABA-inducible promoter, RD29A, to activate its gene expression.

### Example 8

### Deliver Small Activating RNAs in Plant by Using a Ta-siRNA Precursor

Arabidopsis ta-siRNA gene At3g17185 was PCR amplified from Arabidopsis genomic DNA using primers MW-P11F (5′ CCATATCGCAACGATGACGT 3′) and MW-P12R (5′ GCCAGTCCCCTTGATAGCGA 3′) followed by TA cloning into PCR8/GW/TOPO vector (Invitrogen #K2500-20). The 1200 bp of At3g17185 gene contains a 178 bp ta-siRNA region, an 865 bp ta-siRNA upstream region (a potential promoter region) and a 156 bp ta-siRNA downstream region (a potential terminator region). Among the eight 21-nt ta-siRNA phases starting from the position 11 of miR390, two very similar phases, 5′D7(+) and 5′D8(+), are replaced with the same two 21-nt fragments from A-16 (SEQ ID NO: 16). Such engineered ta-siRNA precursors are used as entry vectors for generating binary expression vectors in which expression of ta-siRNA precursor is under the control of Parsley ubiquitin promoter and the 3′UTR of nopaline synthase from Agrobacterium tumefaciens T-DNA (RWT384, SEQ ID NO: 48). RWT 384 produces small activating RNAs targeting RD29A promoter, an ABA-inducible promoter, to activate RD29A gene expression. RWT385 (SEQ ID NO: 49) is made in a similar manner and produces small activating RNA targeting 5′UTR of GH3 gene to activate its expression. Other small activating RNAs can be engineered into ta-siRNA precursors (miR390 or miR173 derived) in a similar manner.

### Example 9

### Whole Plant Transformation

To test RNAa in whole plants, constructs with siRNA hits from the IAA and ABA hormone inducible promoters were transformed into Arabidopsis seedlings. Tansformation was done in Arabidopsis col-0 and ABA2-1 mutants. ABA2-1 mutants were obtained from the Arabidopsis Stock center.

Constructs were designed using siRNAs as described in Example 7. Six weeks old Arabidopsis seedlings of col-0 and ABA2-1 were transformed with these constructs using the flower dip method (Clough and Bent, 1998, Plant J 16:735-43) to generate transgenic lines.

The transgenic lines were grown in the greenhouse and seeds were harvested from these transgenic lines. Leaves from these T1 lines were collected, RNA extracted and qRT-PCR conducted using TaqMan. Ten plants from each construct were used for qRT-PCR. RNAa effect in whole plants was confirmed by up-regulation of GH3 (AT2G23170) in the plants transformed using the IAA siRNA constructs and RD29A (AT5G52310) in the plants transformed using the ABA siRNA constructs. Actin was used as an internal control for normalization. The results were statistically analyzed using the SAS mixed model test for significance at 0.05 confidence level according to which RTP3361, 62, 63, 65 and 68 showed significant upregulation of RD29A. Three to 11 fold up-regulation of AT5G52310 was seen in the ABA constructs (Table 10). Among the IAA siRNA constructs RTP3369, 75 and 76 showed significant upregulation (Table 11).

TABLE 10
Relative expression of RD29A (AT5G52310) in plants transformed using the
ABA siRNA constructs

| | | Construct | | Standard | | | | expression |
|---|---|---|---|---|---|---|---|---|
| construct | siRNA | (control) | Estimate | Error | DF | t Value | Pr > \|t\| | ratio |
| RTP3360 | ABA-1 | RTP3377 | −1.43 | 0.742 | 66 | −1.94 | 0.0556 | 2.71 |
| RTP3361 | ABA-4 | RTP3377 | −3.57 | 0.79 | 66 | −4.5 | 2.69E−05 | 11.86* |
| RTP3362 | ABA-16 | RTP3377 | −2.23 | 0.72 | 66 | −3.11 | 0.0027 | 4.69* |
| RTP3363 | ABA-23 | RTP3377 | −1.74 | 0.74 | 66 | −2.36 | 0.0212 | 3.34* |
| RTP3365 | ABA-27 | RTP3377 | −1.92 | 0.74 | 66 | −2.61 | 0.0113 | 3.79* |
| RTP3366 | ABA-28 | RTP3377 | −0.99 | 0.72 | 66 | −1.38 | 0.1736 | 1.98 |
| RTP3368 | ABA-33 | RTP3377 | −2.47 | 0.72 | 66 | −3.45 | 0.0009 | 5.56* |

*Significant at p < 0.05

TABLE 11
Relative expression of GH3 (AT2G23170) in plants transformed using the
IAA siRNA constructs

| | | construct | | Standard | | | | expression |
|---|---|---|---|---|---|---|---|---|
| construct | siRNA | (control) | Estimate | Error | DF | t Value | Pr > \|t\| | ratio |
| RTP3369 | IAA-24 | 3377 | −5.26 | 0.61 | 137 | −8.58 | 1.84E−14 | 38.21* |
| RTP3370 | IAA-25 | 3377 | −1.52 | 0.6 | 137 | −2.54 | 0.01 | 2.87 |
| RTP3371 | IAA-59 | 3377 | −0.79 | 0.6 | 137 | −1.31 | 0.19 | 1.72 |
| RTP3372 | IAA-75 | 3377 | 1.19 | 0.61 | 137 | 1.94 | 0.05 | 0.44 |
| RTP3373 | IAA-113 | 3377 | −0.56 | 0.6 | 137 | −0.93 | 0.35 | 1.47 |
| RTP3374 | IAA-114 | 3377 | 0.14 | 1.26 | 137 | 0.11 | 0.91 | 0.91 |
| RTP3375 | IAA-115 | 3377 | −2.61 | 0.61 | 137 | −4.26 | 3.84E−05 | 6.09* |
| RTP3376 | IAA-146 | 3377 | −2.23 | 0.61 | 137 | −3.64 | 0.00038 | 4.69* |

*Significant at p < 0.05

### Example 10

### RNAa in Non-Hormone Promoters

Arabidopsis expression profiling was done with affymetric chips using protoplast RNA to select candidate genes for RNAa experiments using a non-hormone promoter. Ten candidate genes were narrowed down based on low to medium expression in the microarray experiments. Two Kb upstream putative promoter regions of these genes were isolated using PCR and cloned with the luciferase reporter and nos terminator. The constructs were transformed into Arabidopsis protoplasts and luciferase assays were conducted as explained in example 1. Based on low to medium luciferase expression, 2 promoters (AT4G36930 and AT2G37590) corresponding to RTP numbers 4044 and 4050 were selected (Table 12). We then designed siRNAs for these 2 promoters starting from 50 bp upstream of the predicted transcription start site of these promoters to the start codon of the gene. A total of 14 and 41 siRNAs were designed for AT4G36930 and AT2G37590 respectively. Arabidopsis protoplasts were transformed with the promoter::reporter constructs and the respective siRNAs and luciferase assays were performed as explained in example 1. We were able to show RNA activation in both promoters tested based on luciferase expression. Six out of the 14 siRNAs tested in the promoter of AT4G36930 (construct RTP4044) showed RNAa effect (Table 13) and 6 out of 41 siRNAs for the promoter AT2G37590 (construct RTP 4050) showed RNAa effect (Table 14).

TABLE 12
Relative Luciferase expression of Arabidopsis RNAa candidates

| | Gene ID of | | |
|---|---|---|---|
| | the | | |
| Construct | promoter | RLU | Std dev |
| No DNA | None | 0.23 | 0.18 |
| DNA no ABA | AT5G52310 | 11.3 | 2.67 |
| DNA + ABA | AT5G52310 | 36.09 | 5.11 |
| RTP 4042 | AT5G15710 | 304.697 | 142.86 |
| RTP4043 | AT4G37480 | 0.97 | 0.078 |
| RTP4044 | AT4G36930 | 58.05 | 12.79 |
| RTP4045 | AT4G26150 | 264.14 | 114.9 |
| RTP4046 | AT3G55170 | 0.79 | 0.72 |
| RTP4047 | AT3G53090 | 2.62 | 1.73 |
| RTP 4049 | AT2G47260 | 298.19 | 44.11 |
| RTP4050 | AT2G37590 | 144.19 | 42.96 |
| RTP4051 | AT2G18350 | 691.45 | 22.67 |
| RTP4052 | AT1G68590 | 7.9 | 1.12 |

TABLE 13
Relative Luciferase Expression of non-hormone promoter of AT4G36930
activated by siRNAs

| | | | | | nucleotide | |
|---|---|---|---|---|---|---|
| siRNA | SEQ ID | | SEQ ID | | positions of | |
| name | NO | Sense sequence | NO | Anti-sense sequence | SEQ ID NO: 236 | |
| NPAT4-1 | 237 | ucucccucucuccaugcccau | 238 | gggcauggagagagggagagu | 1946 | 1966 |
| NPAT4-5 | 239 | uaaaaucucaaagacuguuua | 240 | aacagucuuugagauuuuaug | 1966 | 1986 |
| NPAT4-6 | 241 | ucucaaagacuguuuaaaaaa | 242 | uuuuaaacagucuuugagauu | 1971 | 1991 |
| NPAT4-10 | 243 | aaaaaauguuuuagcuuuaac | 244 | uaaagcuaaaacauuuuuuuu | 1991 | 2011 |
| NPAT4-11 | 245 | auguuuuagcuuuaacugcuu | 246 | gcaguuaaagcuaaaacauuu | 1996 | 2016 |
| NPAT4-13 | 247 | uuuaacugcuuuuuuuuuguu | 248 | caaaaaaaaagcaguuaaagc | 2006 | 2026 |
| siRNA | | | | | | |
| hits | RLU | Std Dev | | | | |
| RTP4044 | 3.62 | 0.61 | | | | |
| (control) | | | | | | |
| NPAT4-1 | 7.21 | 0.09 | | | | |
| NPAT4-5 | 8.31 | 2.06 | | | | |
| NPAT4-6 | 9.73 | 2.27 | | | | |
| NPAT4-10 | 8.49 | 2.4 | | | | |
| NPAT4-11 | 13.59 | 2.94 | | | | |
| NPAT4-13 | 11.08 | 3.31 | | | | |

TABLE 14
Relative Luciferase Expressin of non-hormone promoter of AT2G37590
activated by siRNAs

| | | | | | nucleotide | |
|---|---|---|---|---|---|---|
| siRNA | SEQ ID | | SEQ ID | | positions of | |
| name | NO | Sense sequence | NO | Anti-sense sequence | SEQ ID NO: 249 | |
| NPAT2-5 | 250 | auaaagguucauccacuuuaa | 251 | aaaguggaugaaccuuuauau | 1213 | 1233 |
| NPAT2-6 | 252 | gguucauccacuuuaaauuuu | 253 | aauuuaaaguggaugaaccuu | 1218 | 1238 |
| NPAT2-8 | 254 | cuuuaaauuuuagccaucuuc | 255 | agauggcuaaaauuuaaagug | 1228 | 1248 |
| NPAT2-10 | 256 | uagccaucuucauucucacac | 257 | gugagaaugaagauggcuaaa | 1238 | 1258 |
| NPAT2-11 | 258 | aucuucauucucacacucaac | 259 | ugagugugagaaugaagaugg | 1243 | 1263 |
| NPAT2-18 | 260 | ucauucucauucucucucggc | 261 | cgagagagaaugagaaugaaa | 1278 | 1298 |
| siRNA | | | | | | |
| hits | RLU | Std Dev | | | | |
| RTP4050 | | | | | | |
| (control) | 63.58 | 6.28 | | | | |
| NPAT2-5 | 104.03 | 28.56 | | | | |
| NPAT2-6 | 128.25 | 25.63 | | | | |
| NPAT2-8 | 90.71 | 8.09 | | | | |
| NPAT2-10 | 133.62 | 10.43 | | | | |
| NPAT2-11 | 144.74 | 10.18 | | | | |
| NPAT2-18 | 150.4 | 29.16 | | | | |

### Example 11

### Further Analysis of siRNAs Targeting Hormone-Inducible Promoter

Mutated siRNAs

From the initial experiments nine siRNAs corresponding to regions of the ABA inducible promoter were found to activate gene expression. Eight siRNAs corresponding to regions of the IAA inducible promoter were found to activate gene expression. Make mutations in the siRNAs discovered in the initial ABA and IAA inducible promoter experiments to have the ability to activate gene expression. Specific nucleotide or nucleotides will be changed in the siRNA duplexes to study the effect specific positions have on gene activation.

### Positions 2 and 3

Design siRNAs to test the necessity of positions two and three having a perfect match to their promoter target regions are for RNA induced gene activation. Mutate positions two and three of the sense and antisense strands individually of the functional siRNAs A-29 and A-33. Make the corresponding mutations in the opposite strand of the duplex siRNAs. Maintain the same G/C content as the functional siRNAs when making the mutations. The mutated nucleotides are represented as upper case letters in the table below.

TABLE 15
siRNAs with positions 2 and 3 of the sense and
the antisense strand mutated separately

| Original | Mutated | Sense | Anti-sense |
|---|---|---|---|
| siRNA | siRNA | sequence | sequence |
| A-29 | A29-13 | aUAaugcaaacuag | guuuucuaguuugc |
| | | aaaacaa | auuAUug |
| A-29 | A29-14 | aauaugcaaacuag | gAAuucuaguuugc |
| | | aaUUcaa | auauuug |
| A-33 | A33-13 | aAGaucaggaauaa | acccuuuauuccug |
| | | aggguuu | auCUuug |
| A-33 | A33-14 | aucaucaggaauaa | aGGcuuuauuccug |
| | | agCCuuu | augauug |

### Positions 19 and 20

Design siRNAs to test the necessity of positions 19 and 20 having a perfect match to their promoter target regions are for RNA induced gene activation. Mutate positions 19 and 20 of the sense and antisense strands individually of the functional siRNAs A-29 and A-33. Make the corresponding mutations in the opposite strand of the duplex siRNAs. Maintain the same G/C content as the functional siRNAs when making the mutations. The mutated nucleotides are represented as upper case letters in the table below.

TABLE 16
siRNAs with positions 19 and 20 of the sense
and the antisense strand mutated separately

| Original | Mutated | Sense | Anti-sense |
|---|---|---|---|
| siRNA | siRNA | sequence | sequence |
| A-29 | A29-15 | aauaugcaaacuag | Cuuuucuaguuugc |
| | | aaaaGUa | auauuug |
| A-29 | A29-16 | Uauaugcaaacuag | guuuucuaguuugc |
| | | aaaacaa | auauAAg |
| A-33 | A33-15 | aucaucaggaauaa | Ucccuuuauuccug |
| | | agggAAu | augauug |
| A-33 | A33-16 | Uucaucaggaauaa | acccuuuauuccug |
| | | aggguuu | augaAAg |

Mutations in Only One Strand of siRNAs

Make mutations in the siRNAs discovered in the initial ABA and IAA inducible promoter experiments to have the ability to activate gene expression. Specific nucleotide or nucleotides will be changed in the siRNA duplexes to study the effect specific positions have on gene activation. In previous experiments (see Example 6) we have demonstrated that siRNAs lose the ability to activate transcription when positions 4, 5, and 6 or 16, 17, and 18 are mutated along with their complementary bases. Design siRNAs that contain mutations in only one strand of the duplex siRNAs. The mutated nucleotides are represented as upper case letters in the table below.

TABLE 17
Mutations in only one strand of siRNAs at
positions 4, 5, and 6.

| Original | Mutated | Sense | Anti-sense |
|---|---|---|---|
| siRNA | siRNA | sequence | sequence |
| A-29 | | aauaugcaaacuag | guuuucuaguuugc |
| | | aaaacaa | auauuug |
| A-29 | A29-1 | aauUACcaaacuag | guuuucuaguuugG |
| | | aaaacaa | UAauuug |
| A-29 | A29-2 | aauUACcaaacuag | guuuucuaguuugc |
| | | aaaacaa | auauuug |
| A-29 | A29-3 | aauaugcaaacuag | guuuucuaguuugG |
| | | aaaacaa | UAauuug |
| A-29 | A29-4 | aauaugcaaacuaC | guuAAGuaguuugc |
| | | UUaacaa | auauuug |
| A-29 | A29-5 | aauaugcaaacuag | guuAAGuaguuugc |
| | | aaaacaa | auauuug |
| A-29 | A29-6 | aauaugcaaacuaC | guuuucuaguuugc |
| | | UUaacaa | auauuug |
| A-33 | | aucaucaggaauaa | acccuuuauuccug |
| | | aggguuu | augauug |
| A-33 | A33-1 | aucUAGaggaauaa | acccuuuauuccuC |
| | | aggguuu | UAgauug |
| A-33 | A33-2 | aucUAGaggaauaa | acccuuuauuccug |
| | | aggguuu | augauug |
| A-33 | A33-3 | aucaucaggaauaa | acccuuuauuccuC |
| | | aggguuu | UAgauug |
| A-33 | A33-4 | aucaucaggaauaU | accGAAuauuccug |
| | | UCgguuu | augauug |
| A-33 | A33-5 | aucaucaggaauaa | accGAAuauuccug |
| | | aggguuu | augauug |
| A-33 | A33-6 | aucaucaggaauaU | acccuuuauuccug |
| | | UCgguuu | augauug |

TABLE 18
Mutations in only one strand of siRNAs at
positions 16, 17, and 18.

| | | | |
|---|---|---|---|
| A-29 | A29-7 | aauaugcaaacuag | gAAAucuaguuugc |
| | | aUUUcaa | auauuug |
| A-29 | A29-8 | aauaugcaaacuag | guuuucuaguuugc |
| | | aUUUcaa | auauuug |
| A-29 | A29-9 | aauaugcaaacuag | gAAAucuaguuugc |
| | | aaaacaa | auauuug |
| A-29 | A29-10 | aUAUugcaaacuag | guuuucuaguuugc |
| | | aaaacaa | aAUAuug |
| A-29 | A29-11 | aauaugcaaacuag | guuuucuaguuugc |
| | | aaaacaa | aAUAuug |
| A-29 | A29-12 | aUAUugcaaacuag | guuuucuaguuugc |
| | | aaaacaa | auauuug |
| A-33 | A33-7 | aucaucaggaauaa | aGGGuuuauuccug |
| | | aCCCuuu | augauug |
| A-33 | A33-8 | aucaucaggaauaa | acccuuuauuccug |
| | | aCCCuuu | augauug |
| A-33 | A33-9 | aucaucaggaauaa | aGGGuuuauuccug |
| | | aggguuu | augauug |
| A-33 | A33-10 | aAGUucaggaauaa | acccuuuauuccug |
| | | aggguuu | aACUuug |
| A-33 | A33-11 | aucaucaggaauaa | acccuuuauuccug |
| | | aggguuu | aACUuug |
| A-33 | A33-12 | aAGUucaggaauaa | acccuuuauuccug |
| | | aggguuu | augauug |

Different Length siRNAs

Small RNAs, including siRNAs and miRNAs can range in length from 18 to 24 nucleotides. From the initial experiments nine siRNAs corresponding to regions of the ABA inducible promoter were found to activate gene expression. Design 18 and 24 nucleotide siRNAs based on ABA-29 and ABA-33.

TABLE 19
18 nucleotide siRNAs

| | | | |
|---|---|---|---|
| A-29 | A29-17 | aauaugcaaacuag | uucuaguuugcaua |
| | | aaaa | uuug |
| A-29 | A29-18 | auaugcaaacuaga | uuucuaguuugcau |
| | | aaac | auuu |
| A-29 | A29-19 | uaugcaaacuagaa | uuuucuaguuugca |
| | | aaca | uauu |
| A-29 | A29-20 | augcaaacuagaaa | guuuucuaguuugc |
| | | acaa | auau |
| A-33 | A33-17 | aucaucaggaauaa | cuuuauuccugaug |
| | | aggg | auug |
| A-33 | A33-18 | ucaucaggaauaaa | ccuuuauuccugau |
| | | gggu | gauu |
| A-33 | A33-19 | caucaggaauaaag | cccuuuauuccuga |
| | | gguu | ugau |
| A-33 | A33-20 | aucaggaauaaagg | acccuuuauuccug |
| | | guuu | auga |

TABLE 20
24 nucleotide siRNAs

| | | | |
|---|---|---|---|
| A-29 | A29-21 | aauaugcaaacuagaaa | auuguuuucuaguuugc |
| | | acaauca | auauuug |
| A-29 | A29-22 | acaaauaugcaaacuag | guuuucuaguuugcaua |
| | | aaaacaa | uuuguga |
| A-33 | A33-21 | aucaucaggaauaaagg | caaacccuuuauuccug |
| | | guuugau | augauug |
| A-33 | A33-22 | acaaucaucaggaauaa | acccuuuauuccugaug |
| | | aggguuu | auuguuu |

### Example 12

### RNAa in Monocots

The 2 kb upstream putative promoter region of the Gene GRMZM2G140653 was PCR amplified and cloned with the luciferase reporter and the NOS terminator. The construct was named RTP 4962.

This constructs was then transformed into maize protoplasts as previously described by Hwang and Sheen (2001). RTP4962 showed luciferase expression in protoplast assays. A total of 63 siRNAs were designed to this promoter (GRMZM2G140653) and 34 of them were tested in maize protoplasts. Out of the 34 siRNAs tested 4 showed an activation of 1.5 to 2 fold (Table 21).

TABLE 21
Relative Luciferase expression of Maize GRMZM2G140653 promoter
activated by siRNAs

| siRNA | RLU | Std dev |
|---|---|---|
| RTP4962 | 9.59 | 1.52 |
| NF-3 | 18.09 | 2.46 |
| NF-5 | 16.01 | 2.2 |
| NF-6 | 15.54 | 5.21 |
| NF-34 | 16.38 | 3.44 |

TABLE 22
siRNAs to the GRMZM2G140653-LUC promoter that activated luciferase
expression (with SEQ ID NO)

| siRNA | SEQ ID | | SEQ ID | Anti-sense | nucleotide positions | |
|---|---|---|---|---|---|---|
| name | NO | Sense sequence | NO | sequence | of SEQ ID NO: 262 | |
| NF-3 | 263 | uuuuauaaaauuuga | 264 | uuaaucaaauuuuau | 1728 | 1748 |
| | | uuaaaa | | aaaaua | | |
| NF-5 | 265 | uuugauuaaaacagu | 266 | uuauacuguuuuaau | 1738 | 1758 |
| | | auaaag | | caaauu | | |
| NF-6 | 267 | uuaaaacaguauaaa | 268 | augcuuuauacuguu | 1743 | 1763 |
| | | gcauuu | | uuaauc | | |
| NF-34 | 269 | aauuauaaaguauuu | 270 | auaaaaauacuuuau | 1883 | 1903 |
| | | uuaugu | | aauuua | | |

1. A method for increasing compared to a respective wild-type or part thereof, the expression of a target gene in a plant or part thereof, comprising introducing into said plant or part thereof a recombinant nucleic acid molecule not occurring in a respective wild-type plant or part thereof wherein at least a part of said recombinant nucleic acid molecule is complementary to at least a part of a promoter regulating expression of a target gene in said plant or part thereof.

2. A method as described in claim 1, wherein said recombinant nucleic acid molecule being complementary to at least a part of a promoter regulating expression of a target gene is complementary to a part of said promoter which is 100 bp or less away of the transcription initiation site, or it is complementary to the transcription initiation site of said promoter.

3. A method as described in claim 1 wherein said recombinant nucleic acid molecule being complementary to at least a part of a promoter regulating expression of a target gene is complementary to a part of the promoter which comprises at least a part of a regulatory box of said promoter or which is not more than 100 bp away of such regulatory box.

4. The method as claimed in claim 1, comprising:
a) producing one or more small nucleic acid molecule complementary to a promoter of a target gene,
b) testing said one or more small nucleic acid molecule in vivo or in vitro for their target gene expression increasing property,
c) identifying whether the small nucleic acid molecule increases the target gene expression, and
d) introducing said one or more small nucleic acid molecule into a plant.

5. The method according to claim 4, wherein said small nucleic acid molecules increasing the target gene expression are introduced into said plant by cloning the small nucleic acid molecules increasing the target gene expression into plant transformation vectors comprising plant specific regulatory elements, transforming plants or parts thereof with said vector, and recovering transgenic plants comprising said vector or a part of said vector.

6. The method according to claim 4, wherein said small nucleic acid molecules increasing the target gene expression are introduced into said plant by synthesizing the small nucleic acid molecules increasing the target gene expression and transforming plants or parts thereof with said synthesized small nucleic acid molecules.

7. A method for increasing the expression of a target gene in a plant or part thereof, comprising introducing into said plant or part thereof a recombinant nucleic acid molecule comprising a modified small non-coding RNA, wherein the sequence of said modified small non-coding RNA is modified in relation to a wild-type small non-coding RNA sequence by at least replacing one region of said natural small non-coding RNA complementary to its respective homologous target sequence by a sequence, which is complementary to a promoter regulating expression of a target gene and which is heterologous with regard to said natural small non-coding RNA.

8. A method for identifying small non-coding activating RNAs in a plant or part thereof comprising the steps of
obtaining small RNA molecules from said plant or part thereof,
identifying the sequence of said small RNA molecules,
selecting small RNA molecules comprising regions complementary to at least one promoter of an endogenous gene via bioinformatic analysis and
testing small RNA molecule candidates in a plant or part thereof to determine whether they increase target gene expression.

9. A method for identifying activating microRNAs in a plant or part thereof comprising the steps of
identifying microRNAs in said plant or part thereof being homologous to a promoter in the respective plant,
cloning said microRNAs from said plant or part thereof,
over expressing said microRNAs in a plant and
comparing gene expression in said transgenic plants with respective wild-type plants.

10. A method for replacing the regulatory specificity of a plant specific promoter by modifying in said plant specific promoter a sector targeted by a small non-coding activating RNA conferring activation of expression of genes controlled by said promoter.

11. A method for replacing the regulatory specificity of a plant specific promoter by introducing into said plant specific promoter a sector homologous to a small non-coding activating RNA conferring increase of expression of genes controlled by said promoter.

12. The method of claim 11, wherein said sector is replacing a sector homologous to an endogenous small non-coding activating RNA.

13. The method of claim 11, wherein said sector is homologous to an endogenous small non-coding activating RNA.

14. The method of claim 11, wherein said sector is homologous to a recombinant small non-coding activating RNA.

15. The method of claim 11, wherein the plant specific promoter is modified in vivo.

16. The method of claim 11, wherein the plant specific promoter is modified in vitro.

17. A nucleic acid construct for expression in plants comprising a recombinant nucleic acid molecule comprising a sequence encoding a modified small non-coding RNA sequence, wherein said sequence is modified in relation to a wild-type small non-coding RNA sequence by at least replacing one region of said wild-type small non-coding RNA complementary to its respective wild-type target sequence by a sequence, which is complementary to a promoter regulating expression of a target gene and which is heterologous with regard to said natural small non-coding RNA and which confers increase of expression of said target gene upon introduction into said plant or part thereof.

18. The nucleic acid construct according to claim 17, wherein the transcript of the recombinant nucleic acid molecule is able to form a double stranded structure, wherein said double stranded structure comprises the sequence being complementary to a promoter regulating expression of a target gene.

19. The nucleic acid construct according to claim 18, wherein the double stranded structure is a hairpin structure.

20. The nucleic acid construct according to claim 18, wherein the part of said recombinant nucleic acid molecule being complementary to a promoter regulating expression of a target gene has a length from 15 to 30 bp.

21. The nucleic acid construct according to claim 20, wherein the part of said recombinant nucleic acid molecule being complementary to a promoter regulating expression of a target gene has a length of 19 to 26 bp, 20 to 25 bp, 21 to 24 bp, 21 bp, or 24 bp.

22. The nucleic acid construct according to claim 17, wherein the part of said recombinant nucleic acid molecule being complementary to a promoter regulating expression of a target gene has an identity of 60% or more, 70% or more, 75% or more, 80% or more, 85%, 90%, 91%, 92%, 93%, 94%, 95%, 96%, 97%, 98%, 99% or more, or 100%.

23. The nucleic acid construct according to claim 21, wherein the part of said recombinant nucleic acid molecule being complementary to a promoter regulating expression of a target gene comprises 7 to 11, 8 to 10, or 9 consecutive base pairs homologous, to said target gene promoter.

24. The nucleic acid construct according to claim 23, wherein the part of said recombinant nucleic acid molecule being complementary to a promoter regulating expression of a target gene wherein said consecutive base pairs are at least 80% identical, 90% identical, 95% identical, or 100% identical to said target gene promoter.

25. A vector comprising the nucleic acid construct of claim 17.

26. A system for activating gene expression in a plant or part thereof comprising a) a plant specific promoter comprising a sector homologous to a small non coding activating RNA heterologous to said promoter and b) a construct comprising a small non coding activating RNA homologous to the sector as defined in a) under the control of a plant specific promoter.

27. The system as defined in claim 26 for activating gene expression of an endogenous gene.

28. The system as defined in claim 26 for increasing gene expression of a transgene.

29. A plant or part thereof comprising the recombinant nucleic acid construct of claim 17, wherein said recombinant nucleic acid molecule confers an increase of expression of a target gene in said plant or part thereof compared to a respective plant or part thereof not comprising said recombinant nucleic acid molecule.

30. The plant or part thereof according to claim 29, wherein said recombinant nucleic acid molecule is integrated into the genome of said plant or part thereof.

31. A plant cell comprising the recombinant nucleic acid construct of claim 17, wherein said recombinant nucleic acid molecule confers an increase of expression of a target gene in said plant cell compared to a respective plant cell not comprising said recombinant nucleic acid molecule.

32. The plant cell according to claim 31, wherein said recombinant nucleic acid molecule is integrated into the genome of said plant or part thereof.

33. A microorganism able to transfer nucleic acids to a plant or part of a plant comprising the recombinant nucleic acid construct of claim 17, wherein said recombinant nucleic acid molecule confers upon transfer of said recombinant nucleic acid construct an increase of expression of a target gene in said plant or part of a plant compared to a respective plant or part of a plant not comprising said recombinant nucleic acid molecule.

34. (canceled)

35. A method for production of a plant, part thereof or plant cell, having an increase of expression of a target gene compared to a respective wild-type plant, part thereof or plant cell, comprising introducing the nucleic acid construct of claim 17 into a plant, part thereof or a plant cell.

36. A small non-coding activating RNA conferring an increase of gene expression in a plant or part thereof comprising the sequence of SEQ ID NO: 6, 7, 8, 9, 10, 11, 12, 13, 14, 15, 16, 17, 18, 19, 20, 21, 22, 23, 24, 25, 26, 27, 28, 29, 30 and/or 31.

37. (canceled)

38. The method of claim 1, wherein the target gene is an endogenous target gene.

39. The method of claim 1, wherein the target gene is a transgenic target gene.


SEQUENCE LISTING (270 sequences)

SEQ ID NO: 1 - DNA, 357 bp, Arabidopsis thaliana
cccgaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggacc

SEQ ID NO: 2 - DNA, 3517 bp, Arabidopsis thaliana
ttgtcttgcgcatggagatatcaacagtggtcttaaagactattattgacaacaagtcaacaaacattaaacgtgcacctgtctactaactactaataataataatgttaatgacaaaaagataattacaacatccaatcttcgaattatttagaagaaacatcactttgaacttttgaagtatataaagaaaataatgctcttttatctttctttaatttcttgatatttaaatattataatcaaataacaattgcggatgttaattgtataatactctcctccaaactagcaggcacttagtcaagatttcttaatgttatttaatcgcatggttgagaaaaaataaataataataataataataatatacagctatagaattattgtttatctagaaaacaatttttgggttaaacattcggttaacttgggtatgaaatggtatatatattgttttagcattttgaatcaatataaagaaaacataattctacgtatccaaattctctctcaaaatcttcgaccgaattaacgcaaaccttgaaccattcatcatttccctatgaatgtaatcaaaacagcaatggagttaattttgtactatttattaaaaagttatggaacatacttgaattgcttaagattctaaagaaaatatctgtcggattatagtggccattctttcttttcttttatttttgcaataattagtggccatgccttttccatgcttgatattgaaagttgattcacaactcgtaaaatatatctctccgaacgcagcttcgtctttgaattttgaaatagtttcttctaatcaacaaaacaaaaaaacttttatagataatttggttttagttttacataataccatttaacatcgagtaatgagctcaattagacaaagataatatactgtattatttagaccaagaaaattagtaacgaatttctaacctattcaagtaaaataacagacaagtgggaggaaataactatttcaattaacgtatgcaatactaaggaatagtggtatttaattattaaaaagaagaagaaagaatagtgatatggacatactagaaaattgttgaaatggtccaagtgttcccctaaatgtctaatcaaagataaggcatgacccgtgcgaccagtcacttcctcaatttgttttctttctctcattaggtcatattcatatgcataccgtctttttatcaattacatattcaataattattgttttaggtcgtatattctctttttctattagtattatgtataccagtaatcttatttagtctttattttaaggcttacatgtcaactgatcgccatgactgaatattatcttcggatacctccaagatatcaaataatataatatttgaataatattaatatcataattctgatttttttgtatataaagcaaaaggatacaaatgtatatgcttttcatattttcatctatgtgatgtatattattgatttttcttaccaagataccaccgtattaatttttatatcttattatttattactggtatcgtaagaccaatctctttaacacatcgccaaaaaaaatgtataagagctacattatcagttggcttggattttccttttttctgccttcatcactttgcgcgttaattccttctataatcgtcctcaaatatttttaaattgctatgtgctgaatattttaaattttcataaactttaaagcaacctaataattttctatttctttcaatatatttattgtttttcctatttaccaatttaaataaaatataattttaatacataatatacattaaaaattgaacctcttgctctttcagaagaaaaaaaacaaaaagcaaaaaccctagaattaaatagtactgtcaagttggagtgggtacaggcacgatcgagcaaaacgaaatgtcccatgcatgcctttaattgctttagagttagttcttccaaatcaagaattagttttagttatcaataaataggagtacccactagtagtcgtaccacgacctttcaattcataggacaaacgtcttaccccctgcattattatgcgatatatatgtgaatcatacatcatacatggtgcctaaagaaaatgtgaatcatacatatgaatcttaattattccatacatttaaattatgaaatgcataaaaatgttttcgaaaatgattttataaacatatacgcagaatctcaaagttgctaattgtatactattgctttggtttggttgttaggctaacgtcccatgatccttcggaaagataaatatgcatgacccggagtgatggagcatctaaaattttatggaagctttgctcttaaaaagattaagctgtaattgtataagtatatatagttttaaccaacttgtggttatttttgtagtcgtcgaaatatttattatgggattggaagatcatggtggattgatatgtgtgtcttttctatatatttttaatatttaggtcccattaaatcagtttgtgatttcagaatcctaacatacccttttgagattacttttcctatttatctcgaacataacctcattctaaaactacaacactctattgggacaattctatttggatcaaatttgtgctatagtcatgaatttattactcaaaattaactaaaaagaaatgaacgacacatattttaaatgttttctaggtttaatatcaacttcagatgtggaatattccaaacatgttcgagtttttctattgcaatttcaataataattgatcattgtggacgttttaaatcactagaacattttggcctctcacatgtttcatgattagtctttattttatatacagaattccggattatgagagaaaaaaacacatacttttattaataattacaaatttaaattgaaaatttatctttatgttattacatccaatgaactaatgtcaatgcatggaaagaattacttaggctccgggaacaacaacatgatcccttcacacgcatactctaattcaactaaccaagtcttcttaattctgataagtcaaattgaaaatgtcattaccactgattaaaaaggaatcaaaagaaaaaaagcttttagtattgagtattgaccgtcgctatcggtgacaggcagagtcacaagccaaataaaagggaaccgcgtggtacgtacgggctgacgcctagctgctacggtcatgacaataaattgcccaatcaaagtaacatgccaacgtggcgcagacatatcagtcccacatgtctgcccaaaactagccaaagattacgtgaccgcggtccctcttgtcccctgtctcggtctaacgataacaaaccgagcccacttttatgtcgacgtggaatttggctgacgttggttttctccttcttgccactataaatacaaccccatactcgtcgagtttcaatatctcctcatcatcaaacacaaagtctaatattatcacttacaaataccattttatcc

SEQ ID NO: 3 - DNA, 147 bp, Arabidopsis thaliana
gcttaagagccgcctaagagccgcctaagagccgcctaagagccgcctcgaggatgacgcacaatcccactatccttcgcaagacccttcctctatataaggaagttcatttcatttggagaggacgacctgcaggtcgacggatcc

SEQ ID NO: 4 - DNA, 2399 bp, Arabidopsis thaliana
agctagtgcaggtacataagtcaaaagtcacaacatatagtaagtacatcataagttcatttctcttgaaaacgatctctagtgatgatgcactttttaggtatttattcacatccgagagttttctaaatcacaacattgaaacgtgttcatgaatttaaatcaaaacaaacctgctcatgaatttggggatgatcgttataagcaacgctcttcaaaagctttcgatgggtttttaccatcgatatgcgaatgatgggggaaatcaaacggcatttcttcgggagagagccaagcttctctaaaaactctcgagacttcatcaaaaactaccccattgccttcaggcgtgagatggagcccatcactgcttatagccaacaacaacgagcaagaaccatttataaacaacaaacatgattgaggaggagatgagtgtttgtgaaaaaccaacaacatttcagatcagacttagatacagaccttaggtactttttctgccaatcattggtttcctgcatcttagaccataagttgacacatcgcagaccgagttcctcggccaatgcaacacaatgttgtgcatatacccctgttgtttcgtttgttctctcaggctctttcatagctttctcaccgtagattgatctgatgtaacrcaatcactggctcatttaagaaacatgtcggactaaatgctttgagaaacaaaaatgcaaagaagaaaaggatcataataaagcctactctgcataactttgacgtccagcttcatcaattggtggtggagttataagcacaattagcattgtaggtgaacatttctacaaaacagagtcattagtctttaaaggaatcacacttcaagaaaagtaagtccatgcattagagtggatcaaagaagcatatcaaaaccttcaaatgctgaacaatctttctgacattatctgtgtactcttccaccggcacatgttgtctatcactggttcttcctttgagagctgcatcgtttgcaccgaagaatatcgtcgtagcaacaggaggagacgaagagccctaggcaaaaagacagagtagaacacatactgagataggcacaccaaaacaacacgcactacaagaagagttctcatgatatatgcagatgtaattgccttttaccatctcaaaattcaaatctaaatctttcgtacttcaaccaatccatgacaatgtcataaaaccaacacatcctagaatcatatcaaaaccattcagaactgaaccataacaagcataagcaaaacaaaaaatgatgcacaagagacaataactcaagatctatatccacacagatcatacacacaatcacaacagctcatgaacaaaatcatcccaatgactaccaaaattcgttcgtgtttagatcaaatttcaaatcaacatttgtagactcgagggagggagagagagataaaaagtactgacgagagggaagatgtgatgaagcaagaagagagcccatcgggtgttgtagccgccgtagcctcgaaccacaacatcagccttgcgagagtaagcgtcggcaagagcagatccccaaccgccggacctaaaagactgcgccgtgatcgagtcgccgaacagaactatctccggcctcatctcgctgtctccctccggttcactgagtaaaccgttaactgtattaggaaacttaaagtctcttctgggccaaatcatatgggccgggcctttagtattaatttgttatatttttggtgaatgaaaaggtggtaatctaaagaggttttactaattgtagattaaggatgcgtgtaatgttttagtattagtttatgatttcttgatttcacatttttctgtcttgagcttaagtcaaaacctcaaagtgaacttctacgtagatgaagtattgaagtgtagaagttaaatgcgtgaacttccacaattcaactacaaacacacttttaccaaaaaaaactacaaacacacacaaataatttaaaaataaaaaaagaattctacaagttattgaatatcggtttgggtcggttaaatcttgcatcccattccaaattttctggtttgttcttcgttttgactaaacttgaaccgattggtttgactttatttcggttttttgttttaacttatttaataataaaaatccaccgaactattatttatataaaatataaaataaagattttgaaagcaaattgacaaaatcttaaagatatgcaaaatctctcgatttcattcatattttttccctcttttatttctttctttctttctttctacttatataaatggcccaaccactgccaccatggtttcacatcatatctttctcttcctttttcttccaaatccatcaacattcgttgatcacc

SEQ ID NO: 5 - DNA, 272 bp, Arabidopsis thaliana
gagagaatgatgaaggtgtgtgatgagcaagatggagaagcagggcacgtgcattactagctcatatatacactctcaccacaaatgcgtgtatatatgcggaattttgtgatatagatgtgtgtgtgtgttgagtgtgatgatatggatgagttagttcttcatgtgcccatcttcaccatcatgaccactccaccttggtgacgatgacgacgagggttcaagtgttacgcacgtgggaatatacttatatcgataaacacacacgtgcg

SEQ ID NO: 6 - RNA, 21 nt, Artificial sequence
  Synthetic
aauuccggauuaugagagaaa

SEQ ID NO: 7 - RNA, 21 nt, Artificial sequence
  Synthetic
ucucucauaauccggaauucu

SEQ ID NO: 8 - RNA, 21 nt, Artificial sequence
  Synthetic
cggauuaugagagaaaaaaac

SEQ ID NO: 9 - RNA, 21 nt, Artificial sequence
  Synthetic
uuuuuucucucauaauccgga

SEQ ID NO: 10 - RNA, 21 nt, Artificial sequence
  Synthetic
accaagucuucuuaauucuga

SEQ ID NO: 11 - RNA, 21 nt, Artificial sequence
  Synthetic
agaauuaagaagacuugguua

SEQ ID NO: 12 - RNA, 21 nt, Artificial sequence
  Synthetic
uuuaguauugaguauugaccg

SEQ ID NO: 13 - RNA, 21 nt, Artificial sequence
  Synthetic
gucaauacucaauacuaaaag

SEQ ID NO: 14 - RNA, 21 nt, Artificial sequence
  Synthetic
auuacgugaccgcggucccuc

SEQ ID NO: 15 - RNA, 21 nt, Artificial sequence
  Synthetic
gggaccgcggucacguaaucu

SEQ ID NO: 16 - RNA, 21 nt, Artificial sequence
  Synthetic
gugaccgcggucccucuuguc

SEQ ID NO: 17 - RNA, 21 nt, Artificial sequence
  Synthetic
caagagggaccgcggucacgu

SEQ ID NO: 18 - RNA, 21 nt, Artificial sequence
  Synthetic
cgcggucccucuuguccccug

SEQ ID NO: 19 - RNA, 21 nt, Artificial sequence
  Synthetic
ggggacaagagggaccgcggu

SEQ ID NO: 20 - RNA, 21 nt, Artificial sequence
  Synthetic
acaaagucuaauauuaucacu

SEQ ID NO: 21 - RNA, 21 nt, Artificial sequence
  Synthetic
ugauaauauuagacuuugugu

SEQ ID NO: 22 - RNA, 21 nt, Artificial sequence
  Synthetic
aagaucaagccgacacagaca

SEQ ID NO: 23 - RNA, 21 nt, Artificial sequence
  Synthetic
ucugugucggcuugaucuuuu

SEQ ID NO: 24 - RNA, 21 nt, Artificial sequence
  Synthetic
cagacacgcguagagagcaaa

SEQ ID NO: 25 - RNA, 21 nt, Artificial sequence
  Synthetic
ugcucucuacgcgugucugug

SEQ ID NO: 26 - RNA, 21 nt, Artificial sequence
  Synthetic
cgugucccuuuaucucucuca

SEQ ID NO: 27 - RNA, 21 nt, Artificial sequence
  Synthetic
agagagauaaagggacacgua

SEQ ID NO: 28 - RNA, 21 nt, Artificial sequence
  Synthetic
uuagugagacccuccucuguu

SEQ ID NO: 29 - RNA, 21 nt, Artificial sequence
  Synthetic
cagaggagggucucacuaagu

SEQ ID NO: 30 - RNA, 21 nt, Artificial sequence
  Synthetic
ccuccucuguuuuacucacaa

SEQ ID NO: 31 - RNA, 21 nt, Artificial sequence
  Synthetic
gugaguaaaacagaggagggu

SEQ ID NO: 32 - RNA, 21 nt, Artificial sequence
  Synthetic
uuuacucacaaauaugcaaac

SEQ ID NO: 33 - RNA, 21 nt, Artificial sequence
  Synthetic
uugcauauuugugaguaaaac

SEQ ID NO: 34 - RNA, 21 nt, Artificial sequence
  Synthetic
ucacaaauaugcaaacuagaa

SEQ ID NO: 35 - RNA, 21 nt, Artificial sequence
  Synthetic
cuaguuugcauauuugugagu

SEQ ID NO: 36 - RNA, 21 nt, Artificial sequence
  Synthetic
aauaugcaaacuagaaaacaa

SEQ ID NO: 37 - RNA, 21 nt, Artificial sequence
  Synthetic
guuuucuaguuugcauauuug

SEQ ID NO: 38 - RNA, 21 nt, Artificial sequence
  Synthetic
aucaucaggaauaaaggguuu

SEQ ID NO: 39 - RNA, 21 nt, Artificial sequence
  Synthetic
acccuuuauuccugaugauug

SEQ ID NO: 40 - DNA, 10644 bp, Artificial sequence
  Synthetic
gtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaatacgtgtccctttatctctctcattactagctcatatatacactctcaccacaaatgcgtgtatatatgcggaattttgtgatatagatgtgtgtgtgtgttgagtgtgatgatatggatgagttagttctaagagagataaagggacacgtaatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 41 - protein, 670 aa, Artificial sequence
  Synthetic
MAAATTTTTTSSSISFSTKPSPSSSKSPLPISRFSLPFSLNPNKSSSSSRRRGIKSSSPSSISAVLNTTTNVTTTPSPTKPTKPETFISRFAPDQPRKGADILVEALERQGVETVFAYPGGTSMEIHQALTRSSSIRNVLPRHEQGGVFAAEGYARSSGKPGICIATSGPGATNLVSGLADALLDSVPLVAITGQVPRRMIGTDAFQETPIVEVTRSITKHNYLVMDVEDIPRIIEEAFFLATSGRPGPVLVDVPKDIQQQLAIPNWEQAMRLPGYMSRMPKPPEDSHLEQIVRLISESKKPVLYVGGGCLNSSDELGRFVELTGIPVASTLMGLGSYPCDDELSLHMLGMHGTVYANYAVEHSDLLLAFGVRFDDRVTGKLEAFASRAKIVHIDIDSAEIGKNKTPHVSVCGDVKLALQGMNKVLENRAEELKLDFGVWRNELNVQKQKFPLSFKTFGEAIPPQYAIKVLDELTDGKAIISTGVGQHQMWAAQFYNYKKPRQWLSSGGLGAMGFGLPAAIGASVANPDAIVVDIDGDGSFIMNVQELATIRVENLPVKVLLLNNQHLGMVMQWEDRFYKANRAHTFLGDPAQEDEIFPNMLLFAAACGIPAARVTKKADLREAIQTMLDTPGPYLLDVICPHQEHVLPMIPNGGTFNDVITEGDGRIKY

SEQ ID NO: 42 - DNA, 10644 bp, Artificial sequence
  Synthetic
gtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaaacttagtgagaccctcctctgttttactagctcatatatacactctcaccacaaatgcgtgtatatatgcggaattttgtgatatagatgtgtgtgtgtgttgagtgtgatgatatggatgagttagttcatcagaggagggtctcactaagtatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 43 - protein, 670 aa, Artificial sequence; identical to SEQ ID NO: 41
  Synthetic

SEQ ID NO: 44 - DNA, 10644 bp, Artificial sequence
  Synthetic
gtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaaaccctcctctgttttactcacaattactagctcatatatacactctcaccacaaatgcgtgtatatatgcggaattttgtgatatagatgtgtgtgtgtgttgagtgtgatgatatggatgagttagttctagtgagtaaaacagaggagggtatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 45 - protein, 670 aa, Artificial sequence; identical to SEQ ID NO: 41
  Synthetic

SEQ ID NO: 46 - DNA, 10644 bp, Artificial sequence
  Synthetic
gtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaagttttactcacaaatatgcaaacttactagctcatatatacactctcaccacaaatgcgtgtatatatgcggaattttgtgatatagatgtgtgtgtgtgttgagtgtgatgatatggatgagttagttcgattgcatatttgtgagtaaaacatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 47 - protein, 670 aa, Artificial sequence; identical to SEQ ID NO: 41
  Synthetic

SEQ ID NO: 48 - DNA, 10644 bp, Artificial sequence
  Synthetic
aattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaagcttacaggtataaccgtagtcattactagctcatatatacactctcaccacaaatgcgtgtatatatgcggaattttgtgatatagatgtgtgtgtgtgttgagtgtgatgatatggatgagttagttctaactacggttatacctgtaagcatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtac

SEQ ID NO: 49 - protein, 670 aa, Artificial sequence; identical to SEQ ID NO: 41
  Synthetic

SEQ ID NO: 50 - DNA, 10644 bp, Artificial sequence
  Synthetic
aattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaaacgtgaccgcggtccctcttgtcttactagctcatatatacactctcaccacaaatgcgtgtatatatgcggaattttgtgatatagatgtgtgtgtgtgttgagtgtgatgatatggatgagttagttcgacaagagggaccgcggtcacgtatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtac

SEQ ID NO: 51 - protein, 670 aa, Artificial sequence; identical to SEQ ID NO: 41
  Synthetic

SEQ ID NO: 52 - DNA, 10644 bp, Artificial sequence
  Synthetic
aattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaaaccgcggtccctcttgtcccctgttactagctcatatatacactctcaccacaaatgcgtgtatatatgcggaattttgtgatatagatgtgtgtgtgtgttgagtgtgatgatatggatgagttagttcctggggacaagagggaccgcggtatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtac

SEQ ID NO: 53 - protein, 670 aa, Artificial sequence; identical to SEQ ID NO: 41
  Synthetic

SEQ ID NO: 54 - DNA, 10644 bp, Artificial sequence
  Synthetic
aattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaaaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagggcgcgccgagagaatgatgaaggtgtgtgatgagcaaacacaaagtctaatattatcactttactagctcatatatacactctcaccacaaatgcgtgtatatatgcggaattttgtgatatagatgtgtgtgtgtgttgagtgtgatgatatggatgagttagttcattgataatattagactttgtgtatgaccactccaccttggtgacgatgacgacgagggttcaagtgttacgcacgtgggaatatacttatatcgataaacacacacgtgcgcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtac

SEQ ID NO: 55 - protein, 670 aa, Artificial sequence; identical to SEQ ID NO: 41
  Synthetic

SEQ ID NO: 56 - DNA, 9597 bp, Artificial sequence
  Synthetic
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcaggatcctctagaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcgccggcgtcgacgatatcctgcaggtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggcgcgcctttggttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcagatcagaagcttgctatcaactttgtatagaaaagttggctccgaattcgcccttagcttgactagagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcccggatcaagggcgaattcgacccaagtttgtacaaaaaagcaggctccgaattcgcccttccatatcgcaacgatgacgtcaccaaattcatattttaaaactcgtttcgggcaacgacaacgtcatgatccctcccaaaggtctaattgggccccggcccacaaaggttatcatcttctttcttcttctttgtttattgttgcttttgccttaaaatctcttcttcatcatcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctcttttcgtgtccctttatctctctcacgtgtccctttatctctctcatctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttcttatttgatttttgttgttgttggttatattattgcaacaaaattaaagggggtaaggaaggtctcgctatcaaggggactggcaagcttaagggcgaattcgacccagctttcttgtacaaagtggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccaactttattatacatagttgataagcgatcgcagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatgtgttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtg

SEQ ID NO: 57 - protein, 184 aa, Artificial sequence
  Synthetic
SNLGDGQDRTGRYRQAEVQLPETHVMPVPVLEAGRPQHAAGGISERLVHAHARVVGQPDDSDHALEALCLQGLQQVGVERGAQSRPLVAGGDVHGRLGRPVVGVACLPGARVGDAGDLAVHLGDEPGIALPQTDEVVRPLLRFLRLGTEVDRACLDVVVDDGADRRHVRLGGTADVGRASFWAH

SEQ ID NO: 58 - DNA, 9597 bp, Artificial sequence
  Synthetic
gtgattttgtgccgagctgccggtcggggagctgttggctggctggtggcaggatatattgtggtgtaaacaaattgacgcttagacaacttaataacacattgcggacgtctttaatgtactgaattaacatccgtttgatacttgtctaaaattggctgatttcgagtgcatctatgcataaaaacaatctaatgacaattattaccaagcaggatcctctagaattcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagctcgccggcgtcgacgatatcctgcaggtcaaatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggcgcgcctttggttgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcagatcagaagcttgctatcaactttgtatagaaaagttggctccgaattcgcccttagcttgactagagaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagcccggatcaagggcgaattcgacccaagtttgtacaaaaaagcaggctccgaattcgcccttccatatcgcaacgatgacgtcaccaaattcatattttaaaactcgtttcgggcaacgacaacgtcatgatccctcccaaaggtctaattgggccccggcccacaaaggttatcatcttctttcttcttctttgtttattgttgcttttgccttaaaatctcttcttcatcatcccaccgtttcttaagactctctctctttctgttttctatttctctctctctcaaatgaaagagagagaagagctcccatggatgaaattagcgagaccgaagtttctccaaggtgatatgtctatctgtatatgtgatacgaagagttagggttttgtcatttcgaagtcaatttttgtttgtttgtcaataatgatatctgaatgatgaagaacacgtaactaagatatgttactgaactatataatacatatgtgtgtttttctgtatctatttctatatatatgtagatgtagtgtaagtctgttatatagacattattcatgtgtacatgcattataccaacataaatttgtatcaatactacttttgatttacgatgatggatgttcttagatatcttcatacgtttgtttccacatgtatttacaactacatatatatttggaatcacatatatacttgattattatagttgtaaagagtaacaagttcttttttcaggcattaaggaaaacataacctccgtgatgcatagagattattggatccgctgtgctgagacattgagtttttcttcggcattccagtttcaatgataaagcggtgttatcctatctgagcttttagtcggattttttcttttcaattattgtgttttatctagatgatgcatttcattattctcttttgtgaccgcggtccctcttgtcgtgaccgcggtccctcttgtctctctttctaaacgttttattattttctcgttttacagattctattctatctcttctcaatatagaatagatatctatctctacctctaattcgttcgagtcattttctcctaccttgtctatccctcctgagctaatctccacatatatcttttgtttgttattgatgtatggttgacataaattcaataaagaagttgacgtttttcttatttgatttttgttgttgttggttatattattgcaacaaaattaaagggggtaaggaaggtctcgctatcaaggggactggcaagcttaagggcgaattcgacccagctttcttgtacaaagtggagctcgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccaactttattatacatagttgataagcgatcgcagcttggcgtaatcatggtcatagctgtttcctactagatctgattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtccatgtgttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattgaaaaaggaagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtaacgcgtg

SEQ ID NO: 59 - protein, 184 aa, Artificial sequence; identical to SEQ ID NO: 57
  Synthetic

SEQ ID NO: 60 - RNA, 21 nt, Artificial sequence
  Synthetic
uuauuuuauauacagaauucc

SEQ ID NO: 61 - RNA, 21 nt, Artificial sequence
  Synthetic
aauucuguauauaaaauaaag

SEQ ID NO: 62 - RNA, 21 nt, Artificial sequence
  Synthetic
uuauauacagaauuccggauu

SEQ ID NO: 63 - RNA, 21 nt, Artificial sequence
  Synthetic
uuauauacagaauuccggauu

SEQ ID NO: 64 - RNA, 21 nt, Artificial sequence
  Synthetic
uacagaauuccggauuaugag

SEQ ID NO: 65 - RNA, 21 nt, Artificial sequence
  Synthetic
cauaauccggaauucuguaua

SEQ ID NO: 66 - RNA, 21 nt, Artificial sequence
  Synthetic
uauugaguauugaccgucgcu

SEQ ID NO: 67 - RNA, 21 nt, Artificial sequence
  Synthetic
cgacggucaauacucaauacu

SEQ ID NO: 68 - RNA, 21 nt, Artificial sequence
  Synthetic
caaagauuacgugaccgcggu

SEQ ID NO: 69 - RNA, 21 nt, Artificial sequence
  Synthetic
cgcggucacguaaucuuuggc

SEQ ID NO: 70 - RNA, 21 nt, Artificial sequence
  Synthetic
ucccucuuguccccugucucg

SEQ ID NO: 71 - RNA, 21 nt, Artificial sequence
  Synthetic
agacaggggacaagagggacc

SEQ ID NO: 72 - RNA, 21 nt, Artificial sequence
  Synthetic
cucuauaaacuuagugagacc

SEQ ID NO: 73 - RNA, 21 nt, Artificial sequence
  Synthetic
ucucacuaaguuuauagagag

SEQ ID NO: 74 - RNA, 21 nt, Artificial sequence
  Synthetic
uaaacuuagugagacccuccu

SEQ ID NO: 75 - RNA, 21 nt, Artificial sequence
  Synthetic
gagggucucacuaaguuuaua

SEQ ID NO: 76 - RNA, 21 nt, Artificial sequence
  Synthetic
uuagugaguggcuccucuguu

SEQ ID NO: 77 - RNA, 21 nt, Artificial sequence
  Synthetic
cagaggagccacucacuaagu

SEQ ID NO: 78 - RNA, 21 nt, Artificial sequence
  Synthetic
ccuccucucaauuacucacaa

SEQ ID NO: 79 - RNA, 21 nt, Artificial sequence
  Synthetic
gugaguaauugagaggagggu

SEQ ID NO: 80 - RNA, 21 nt, Artificial sequence
  Synthetic
uuuacucaguuauaugcaaac

SEQ ID NO: 81 - RNA, 21 nt, Artificial sequence
  Synthetic
uugcauauaacugaguaaaac

SEQ ID NO: 82 - RNA, 21 nt, Artificial sequence
  Synthetic
ucacaaauuaccaaacuagaa

SEQ ID NO: 83 - RNA, 21 nt, Artificial sequence
  Synthetic
cuaguuugguaauuugugagu

SEQ ID NO: 84 - RNA, 21 nt, Artificial sequence
  Synthetic
aauaugcauuguagaaaacaa

SEQ ID NO: 85 - RNA, 21 nt, Artificial sequence
  Synthetic
guuuucuacaaugcauauuug

SEQ ID NO: 86 - RNA, 21 nt, Artificial sequence
  Synthetic
aucaucagcuuuaaaggguuu

SEQ ID NO: 87 - RNA, 21 nt, Artificial sequence
  Synthetic
acccuuuaaagcugaugauug

SEQ ID NO: 88 - RNA, 21 nt, Artificial sequence
  Synthetic
aauuccgguaaaugagagaaa

SEQ ID NO: 89 - RNA, 21 nt, Artificial sequence
  Synthetic
ucucucauuuaccggaauucu

SEQ ID NO: 90 - RNA, 21 nt, Artificial sequence
  Synthetic
cggauuaucucagaaaaaaac

SEQ ID NO: 91 - RNA, 21 nt, Artificial sequence
  Synthetic
uuuuuucugagauaauccgga

SEQ ID NO: 92 - RNA, 21 nt, Artificial sequence
  Synthetic
auuacgugugggcggucccuc

SEQ ID NO: 93 - RNA, 21 nt, Artificial sequence
  Synthetic
gggaccgcccacacguaaucu

SEQ ID NO: 94 - RNA, 21 nt, Artificial sequence
  Synthetic
gugaccgcccacccucuuguc

SEQ ID NO: 95 - RNA, 21 nt, Artificial sequence
  Synthetic
caagagggugggcggucacgu

SEQ ID NO: 96 - RNA, 21 nt, Artificial sequence
  Synthetic
cgcgguccgaguuguccccug

SEQ ID NO: 97 - RNA, 21 nt, Artificial sequence
  Synthetic
ggggacaacucggaccgcggu

SEQ ID NO: 98 - RNA, 21 nt, Artificial sequence
  Synthetic
uuuugacacaaauaugcaaac

SEQ ID NO: 99 - RNA, 21 nt, Artificial sequence
  Synthetic
uugcauauuugugucaaaaac

SEQ ID NO: 100 - RNA, 21 nt, Artificial sequence
  Synthetic
uuuacucacaaauuaccaaac

SEQ ID NO: 101 - RNA, 21 nt, Artificial sequence
  Synthetic
uugguaauuugugaguaaaac

SEQ ID NO: 102 - RNA, 21 nt, Artificial sequence
  Synthetic
ucaguuauaugcaaacuagaa

SEQ ID NO: 103 - RNA, 21 nt, Artificial sequence
  Synthetic
cuaguuugcauauaacugagu

SEQ ID NO: 104 - RNA, 21 nt, Artificial sequence
  Synthetic
ucacaaauaugcauuguagaa

SEQ ID NO: 105 - RNA, 21 nt, Artificial sequence
  Synthetic
cuacaaugcauauuugugagu

SEQ ID NO: 106 - RNA, 21 nt, Artificial sequence
  Synthetic
auuugcugaccgcggucccuc

SEQ ID NO: 107 - RNA, 21 nt, Artificial sequence
  Synthetic
gggaccgcggucagcaaaucu

SEQ ID NO: 108 - RNA, 21 nt, Artificial sequence
  Synthetic
auuacgugaccgcccacccuc

SEQ ID NO: 109 - RNA, 21 nt, Artificial sequence
  Synthetic
gggugggcggucacguaaucu

SEQ ID NO: 110 - RNA, 21 nt, Artificial sequence
  Synthetic
gugugggcggucccucuuguc

SEQ ID NO: 111 - RNA, 21 nt, Artificial sequence
  Synthetic
caagagggaccgcccacacgu

SEQ ID NO: 112 - RNA, 21 nt, Artificial sequence
  Synthetic
gugaccgcgguccgaguuguc

SEQ ID NO: 113 - RNA, 21 nt, Artificial sequence
  Synthetic
caacucggaccgcggucacgu

SEQ ID NO: 114 - RNA, 21 nt, Artificial sequence
  Synthetic
uuuacucacaaauaucguaac

SEQ ID NO: 115 - RNA, 21 nt, Artificial sequence
  Synthetic
uacgauauuugugaguaaaac

SEQ ID NO: 116 - RNA, 21 nt, Artificial sequence
  Synthetic
uaaucucacaaauaugcaaac

SEQ ID NO: 117 - RNA, 21 nt, Artificial sequence
  Synthetic
uugcauauuugugagauuaac

SEQ ID NO: 118 - RNA, 21 nt, Artificial sequence
  Synthetic
ucacaaauaugcaaagaugaa

SEQ ID NO: 119 - RNA, 21 nt, Artificial sequence
  Synthetic
caucuuugcauauuugugagu

SEQ ID NO: 120 - RNA, 21 nt, Artificial sequence
  Synthetic
ugugaaauaugcaaacuagaa

SEQ ID NO: 121 - RNA, 21 nt, Artificial sequence
  Synthetic
cuaguuugcauauuucacagu

SEQ ID NO: 122 - RNA, 21 nt, Artificial sequence
  Synthetic
auuacgugaccgcggaggcuc

SEQ ID NO: 123 - RNA, 21 nt, Artificial sequence
  Synthetic
gccuccgcggucacguaaucu

SEQ ID NO: 124 - RNA, 21 nt, Artificial sequence
  Synthetic
aaaucgugaccgcggucccuc

SEQ ID NO: 125 - RNA, 21 nt, Artificial sequence
  Synthetic
gggaccgcggucacgauuucu

SEQ ID NO: 126 - RNA, 21 nt, Artificial sequence
  Synthetic
gugaccgcggucccugaaguc

SEQ ID NO: 127 - RNA, 21 nt, Artificial sequence
  Synthetic
cuucagggaccgcggucacgu

SEQ ID NO: 128 - RNA, 21 nt, Artificial sequence
  Synthetic
gacuccgcggucccucuuguc

SEQ ID NO: 129 - RNA, 21 nt, Artificial sequence
  Synthetic
caagagggaccgcggagucgu

SEQ ID NO: 130 - RNA, 21 nt, Artificial sequence
  Synthetic
auuacucacaaauaugcaaac

SEQ ID NO: 131 - RNA, 21 nt, Artificial sequence
  Synthetic
uugcauauuugugaguaauac

SEQ ID NO: 132 - RNA, 21 nt, Artificial sequence
  Synthetic
guuacucacaaauaugcaaac

SEQ ID NO: 133 - RNA, 21 nt, Artificial sequence
  Synthetic
uugcauauuugugaguaacac

SEQ ID NO: 134 - RNA, 21 nt, Artificial sequence
  Synthetic
cuuacucacaaauaugcaaac

SEQ ID NO: 135 - RNA, 21 nt, Artificial sequence
  Synthetic
uugcauauuugugaguaagac

SEQ ID NO: 136 - RNA, 21 nt, Artificial sequence
  Synthetic
uuuacucacaaauaugcauac

SEQ ID NO: 137 - RNA, 21 nt, Artificial sequence
  Synthetic
augcauauuugugaguaaaac

SEQ ID NO: 138 - RNA, 21 nt, Artificial sequence
  Synthetic
uuuacucacaaauaugcacac

SEQ ID NO: 139 - RNA, 21 nt, Artificial sequence
  Synthetic
gugcauauuugugaguaaaac

SEQ ID NO: 140 - RNA, 21 nt, Artificial sequence
  Synthetic
uuuacucacaaauaugcagac

SEQ ID NO: 141 - RNA, 21 nt, Artificial sequence
  Synthetic
cugcauauuugugaguaaaac

SEQ ID NO: 142 - RNA, 21 nt, Artificial sequence
  Synthetic
cugcauauuugugaguaaaac

SEQ ID NO: 143 - RNA, 21 nt, Artificial sequence
  Synthetic
cuaguuugcauauuugugugu

SEQ ID NO: 144 - RNA, 21 nt, Artificial sequence
  Synthetic
gcacaaauaugcaaacuagaa

SEQ ID NO: 145 - RNA, 21 nt, Artificial sequence
  Synthetic
cuaguuugcauauuugugcgu

SEQ ID NO: 146 - RNA, 21 nt, Artificial sequence
  Synthetic
ccacaaauaugcaaacuagaa

SEQ ID NO: 147 - RNA, 21 nt, Artificial sequence
  Synthetic
cuaguuugcauauuugugggu

SEQ ID NO: 148 - RNA, 21 nt, Artificial sequence
  Synthetic
ucacaaauaugcaaacuauaa

SEQ ID NO: 149 - RNA, 21 nt, Artificial sequence
  Synthetic
auaguuugcauauuugugagu

SEQ ID NO: 150 - RNA, 21 nt, Artificial sequence
  Synthetic
ucacaaauaugcaaacuaaaa

SEQ ID NO: 151 - RNA, 21 nt, Artificial sequence
  Synthetic
uuaguuugcauauuugugagu

SEQ ID NO: 152 - RNA, 21 nt, Artificial sequence
  Synthetic
ucacaaauaugcaaacuacaa

SEQ ID NO: 153 - RNA, 21 nt, Artificial sequence
  Synthetic
guaguuugcauauuugugagu

SEQ ID NO: 154 - RNA, 21 nt, Artificial sequence
  Synthetic
uuuacgugaccgcggucccuc

SEQ ID NO: 155 - RNA, 21 nt, Artificial sequence
  Synthetic
gggaccgcggucacguaaacu

SEQ ID NO: 156 - RNA, 21 nt, Artificial sequence
  Synthetic
guuacgugaccgcggucccuc

SEQ ID NO: 157 - RNA, 21 nt, Artificial sequence
  Synthetic
gggaccgcggucacguaaccu

SEQ ID NO: 158 - RNA, 21 nt, Artificial sequence
  Synthetic
cuuacgugaccgcggucccuc

SEQ ID NO: 159 - RNA, 21 nt, Artificial sequence
  Synthetic
gggaccgcggucacguaagcu

SEQ ID NO: 160 - RNA, 21 nt, Artificial sequence
  Synthetic
auuacgugaccgcgguccuuc

SEQ ID NO: 161 - RNA, 21 nt, Artificial sequence
  Synthetic
aggaccgcggucacguaaucu

SEQ ID NO: 162 - RNA, 21 nt, Artificial sequence
  Synthetic
auuacgugaccgcgguccauc

SEQ ID NO: 163 - RNA, 21 nt, Artificial sequence
  Synthetic
uggaccgcggucacguaaucu

SEQ ID NO: 164 - RNA, 21 nt, Artificial sequence
  Synthetic
auuacgugaccgcgguccguc

SEQ ID NO: 165 - RNA, 21 nt, Artificial sequence
  Synthetic
cggaccgcggucacguaaucu

SEQ ID NO: 166 - RNA, 21 nt, Artificial sequence
  Synthetic
augaccgcggucccucuuguc

SEQ ID NO: 167 - RNA, 21 nt, Artificial sequence
  Synthetic
caagagggaccgcggucaugu

SEQ ID NO: 168 - RNA, 21 nt, Artificial sequence
  Synthetic
uugaccgcggucccucuuguc

SEQ ID NO: 169 - RNA, 21 nt, Artificial sequence
  Synthetic
caagagggaccgcggucaagu

SEQ ID NO: 170 - RNA, 21 nt, Artificial sequence
  Synthetic
cugaccgcggucccucuuguc

SEQ ID NO: 171 - RNA, 21 nt, Artificial sequence
  Synthetic
caagagggaccgcggucaggu

SEQ ID NO: 172 - RNA, 21 nt, Artificial sequence
  Synthetic
gugaccgcggucccucuuuuc

SEQ ID NO: 173 - RNA, 21 nt, Artificial sequence
  Synthetic
aaagagggaccgcggucacgu

SEQ ID NO: 174 - RNA, 21 nt, Artificial sequence
  Synthetic
gugaccgcggucccucuuauc

SEQ ID NO: 175 - RNA, 21 nt, Artificial sequence
  Synthetic
uaagagggaccgcggucacgu

SEQ ID NO: 176 - RNA, 21 nt, Artificial sequence
  Synthetic
gugaccgcggucccucuucuc

SEQ ID NO: 177 - RNA, 21 nt, Artificial sequence
  Synthetic
gaagagggaccgcggucacgu

SEQ ID NO: 178 - DNA, 21 bp, Artificial sequence
  Synthetic
uuuacucacaaauaugcaatt

SEQ ID NO: 179 - DNA, 21 bp, Artificial sequence
  Synthetic
uugcauauuugugaguaaatt

SEQ ID NO: 180 - DNA, 21 bp, Artificial sequence
  Synthetic
ucacaaauaugcaaacuagtt

SEQ ID NO: 181 - DNA, 21 bp, Artificial sequence
  Synthetic
cuaguuugcauauuugugatt

SEQ ID NO: 182 - DNA, 21 bp, Artificial sequence
  Synthetic
auuacgugaccgcgguccctt

SEQ ID NO: 183 - DNA, 21 bp, Artificial sequence
  Synthetic
gggaccgcggucacguaautt

SEQ ID NO: 184 - DNA, 21 bp, Artificial sequence
  Synthetic
gugaccgcggucccucuugtt

SEQ ID NO: 185 - DNA, 21 bp, Artificial sequence
  Synthetic
caagagggaccgcggucactt

SEQ ID NO: 186 - RNA, 21 nt, Artificial sequence
  Synthetic
uacuaauaauaguaaguuaca

SEQ ID NO: 187 - RNA, 21 nt, Artificial sequence
  Synthetic
uaacuuacuauuauuaguagu

SEQ ID NO: 188 - RNA, 21 nt, Artificial sequence
  Synthetic
auaauaguaaguuacauuuua

SEQ ID NO: 189 - RNA, 21 nt, Artificial sequence
  Synthetic
aaauguaacuuacuauuauua

SEQ ID NO: 190 - RNA, 21 nt, Artificial sequence
  Synthetic
ugacuuugacgucacaccacg

SEQ ID NO: 191 - RNA, 21 nt, Artificial sequence
  Synthetic
uggugugacgucaaagucauu

SEQ ID NO: 192 - RNA, 21 nt, Artificial sequence
  Synthetic
aaaugacuuugacgucacacc

SEQ ID NO: 193 - RNA, 21 nt, Artificial sequence
  Synthetic
ugugacgucaaagucauuuug

SEQ ID NO: 194 - RNA, 21 nt, Artificial sequence
  Synthetic
acuuugacgucacaccacgaa

SEQ ID NO: 195 - RNA, 21 nt, Artificial sequence
  Synthetic
cguggugugacgucaaaguca

SEQ ID NO: 196 - RNA, 21 nt, Artificial sequence
  Synthetic
ugacuuugacgucacaccacg

SEQ ID NO: 197 - RNA, 21 nt, Artificial sequence
  Synthetic
uggugugacgucaaagucauu

SEQ ID NO: 198 - RNA, 21 nt, Artificial sequence
  Synthetic
ugacgucacaccacgaaaaca

SEQ ID NO: 199 - RNA, 21 nt, Artificial sequence
  Synthetic
uuuucguggugugacgucaaa

SEQ ID NO: 200 - RNA, 21 nt, Artificial sequence
  Synthetic
cgucacaccacgaaaacagac

SEQ ID NO: 201 - RNA, 21 nt, Artificial sequence
  Synthetic
gucuuuucguggugugacguc

SEQ ID NO: 202 - RNA, 21 nt, Artificial sequence
  Synthetic
gcuucauacgugucccuuuau

SEQ ID NO: 203 - RNA, 21 nt, Artificial sequence
  Synthetic
aaagggacacguaugaagcgu

SEQ ID NO: 204 - RNA, 21 nt, Artificial sequence
  Synthetic
acgcuucauacgugucccuuu

SEQ ID NO: 205 - RNA, 21 nt, Artificial sequence
  Synthetic
agggacacguaugaagcgucu

SEQ ID NO: 206 - RNA, 21 nt, Artificial sequence
  Synthetic
auguauauuauugauuuuucu

SEQ ID NO: 207 - RNA, 21 nt, Artificial sequence
  Synthetic
aaaaaucaauaauauacauca

SEQ ID NO: 208 - RNA, 21 nt, Artificial sequence
  Synthetic
uguauauuauugauuuuucuu

SEQ ID NO: 209 - RNA, 21 nt, Artificial sequence
  Synthetic
gaaaaaucaauaauauacauc

SEQ ID NO: 210 - RNA, 21 nt, Artificial sequence
  Synthetic
uuaucaauaaauaggaguacc

SEQ ID NO: 211 - RNA, 21 nt, Artificial sequence
  Synthetic
uacuccuauuuauugauaacu

SEQ ID NO: 212 - RNA, 21 nt, Artificial sequence
  Synthetic
guuuucgaaaaugauuuuaua

SEQ ID NO: 213 - RNA, 21 nt, Artificial sequence
  Synthetic
uaaaaucauuuucgaaaacau

SEQ ID NO: 214 - RNA, 21 nt, Artificial sequence
  Synthetic
uuuucgaaaaugauuuuauaa

SEQ ID NO: 215 - RNA, 21 nt, Artificial sequence
  Synthetic
auaaaaucauuuucgaaaaca

SEQ ID NO: 216 - RNA, 21 nt, Artificial sequence
  Synthetic
gaauuuauuacucaaaauuaa

SEQ ID NO: 217 - RNA, 21 nt, Artificial sequence
  Synthetic
aauuuugaguaauaaauucau

SEQ ID NO: 218 - RNA, 21 nt, Artificial sequence
  Synthetic
gucaugaauuuauuacucaaa

SEQ ID NO: 219 - RNA, 21 nt, Artificial sequence
  Synthetic
ugaguaauaaauucaugacua

SEQ ID NO: 220 - RNA, 21 nt, Artificial sequence
  Synthetic
cggucaugacaauaaauugcc

SEQ ID NO: 221 - RNA, 21 nt, Artificial sequence
  Synthetic
caauuuauugucaugaccgua

SEQ ID NO: 222 - RNA, 21 nt, Artificial sequence
  Synthetic
augacaauaaauugcccaauc

SEQ ID NO: 223 - RNA, 21 nt, Artificial sequence
  Synthetic
uugggcaauuuauugucauga

SEQ ID NO: 224 - RNA, 21 nt, Artificial sequence
  Synthetic
aaagauuacgugaccgcgguc

SEQ ID NO: 225 - RNA, 21 nt, Artificial sequence
  Synthetic
ccgcggucacguaaucuuugg

SEQ ID NO: 226 - RNA, 21 nt, Artificial sequence
  Synthetic
ccaaagauuacgugaccgcgg

SEQ ID NO: 227 - RNA, 21 nt, Artificial sequence
  Synthetic
gcggucacguaaucuuuggcu

SEQ ID NO: 228 - RNA, 21 nt, Artificial sequence
  Synthetic
ucuuguccccugucucggucu

SEQ ID NO: 229 - RNA, 21 nt, Artificial sequence
  Synthetic
accgagacaggggacaagagg

SEQ ID NO: 230 - RNA, 21 nt, Artificial sequence
  Synthetic
ucccucuuguccccugucucg

SEQ ID NO: 231 - RNA, 21 nt, Artificial sequence
  Synthetic
agacaggggacaagagggacc

SEQ ID NO: 232 - RNA, 21 nt, Artificial sequence
  Synthetic
uaugucgacguggaauuuggc

SEQ ID NO: 233 - RNA, 21 nt, Artificial sequence
  Synthetic
caaauuccacgucgacauaaa

SEQ ID NO: 234 - RNA, 21 nt, Artificial sequence
  Synthetic
uuuaugucgacguggaauuug

SEQ ID NO: 235 - RNA, 21 nt, Artificial sequence
  Synthetic
aauuccacgucgacauaaaag

SEQ ID NO: 236 - DNA, 2035 bp, Arabidopsis thaliana
atagcgtgagtttgatttatgttatccttgttatggtgcatatgtattacggaaactttggtctgttttagatgggtaaatagttgtatatgagtaaatatgatattgccagtgttttatataaagaaaaagggaaacgatgataaaaggtgaaaaagaagtttaggatcatctttgttttttattttgttttccgactttcaaatcagacaagacaacgaggtatgggtcttttaacatacagagactcaacttgaaaattctatcaaaccaccaattaaaaaaccagagaaaacattcaactatcttcttagaatctagacaaaataaaaagtgtatcatgctgattttgaacaaattattaaagagatctcaaccatcgtttcattaatatctttgacaattctagaagccgtagtaaatcttgtctttttatgtgtatatcttgttgaatttgataaggatattcaaaataatgagatatgtatgttgagtaattgagtaaaatcagatcccttgtttaatttgacgattaaacgcttttttcattattttttttcgcattttgttgtgtatgttctcaattacaaattccactataagcatttggactctacaatgtcactttctaattctgatatttaataaaacaattattttgtatatgtataaatagtaccagaaaaacgaacttaataaaataattgaatcaaaagcagatgctgaacaaatagctgcagcttttagcaattgtctcccatatgcttttcctttgtttcaaaaattgtatatacgaggttaagtgaatgttgcaatgaaataaatgatgcagtttgtgcattcatcaagcgaacaagtacgattgatgttttcagtgcaaaaactaaaataaaatactactaacaactatatagtgtaaatatatataactttaacttctttttttttaagtcttcatacgctgcgaacgatcaaaatatatttacaaatttacgaccaatattttaaaaatacttcattaaagaattgatgctgctgtctgcacagttgagaacgcaattggaaactttcgtgcattcactttgttcgccgcaaattggatatgtgaacgtggtcatcattatttatagaattattctataaaacttactataaaacactagatataaagctggatcatataaaatgaatttactaaaaattcgatagttaattatagcagatgttatgttccaatttgaaagcatacgaacgaggtatatagttgaaaacacacaccgtaaagttaataattttcacaacacaagaacaaatcaaagtcgcaagtaatttaacgcatgtcagtagcatgggcttcttatatactagtcaatagaataaactagcaaacaagcaactaatgtattcttgtttatcacgccacgggttacaatatcatacaaaatttgaatactaattaatggtaacaagtaaaaaaacaaattactagaaatggaaatacttttgtcaaacaaccaagacgtataactttcgttttctatagattaatggacttcttaaaaatctctccatcagattaaactttgagatatacaaatacagtttttgttttcttctaaatgatatgaatattaactttatcgatttcatccgtagcagatttccattttaaataataaactatgagaaaacagataaaggttgtatattatttgttacccccaaaaaaaaaaaactaactacgagtagtagtttagtgtgtctcacgtgcgacgaggaaaagttttgggagagtaaaaacatttaatatttacgactagtttgaaaaaccgtgagctgacacaagctcattgctaatgctacagtaacagctaccttcacttttaactaaatgacagaccaatcattttaacctctgttttcttagctggcgcgtgacagacactctccctctctccatgcccataaaatctcaaagactgtttaaaaaaaaaaatgttttagctttaactgctttttttttgttgttggtgta

SEQ ID NO: 237 - RNA, 21 nt, Artificial sequence
  Synthetic
ucucccucucuccaugcccau

SEQ ID NO: 238 - RNA, 21 nt, Artificial sequence
  Synthetic
gggcauggagagagggagagu

SEQ ID NO: 239 - RNA, 21 nt, Artificial sequence
  Synthetis
uaaaaucucaaagacuguuua

SEQ ID NO: 240 - RNA, 21 nt, Artificial sequence
  Synthetic
aacagucuuugagauuuuaug

SEQ ID NO: 241 - RNA, 21 nt, Artificial sequence
  Synthetic
ucucaaagacuguuuaaaaaa

SEQ ID NO: 242 - RNA, 21 nt, Artificial sequence
  Synthetic
uuuuaaacagucuuugagauu

SEQ ID NO: 243 - RNA, 21 nt, Artificial sequence
  Synthetic
aaaaaauguuuuagcuuuaac

SEQ ID NO: 244 - RNA, 21 nt, Artificial sequence
  Synthetic
uaaagcuaaaacauuuuuuuu

SEQ ID NO: 245 - RNA, 21 nt, Artificial sequence
  Synthetic
auguuuuagcuuuaacugcuu

SEQ ID NO: 246 - RNA, 21 nt, Artificial sequence
  Synthetic
gcaguuaaagcuaaaacauuu

SEQ ID NO: 247 - RNA, 21 nt, Artificial sequence
  Synthetic
uuuaacugcuuuuuuuuuguu

SEQ ID NO: 248 - RNA, 21 nt, Artificial sequence
  Synthetic
caaaaaaaaagcaguuaaagc

SEQ ID NO: 249 - DNA, 1413 bp, Arabidopsis thaliana
aatgtaaagtgttctacaaataatcaaaaagttagaaatgatactcgtatcctaatgttttgtattccaaatgtatccaaaactgatcaaaactatcgaaatctaaagtaaaaagtaaaaatcaaaccaccatcgataatcgaatgaccaaatatttaaatctgcaaatagttttgtttgccgtcttttgtttactagattgatgaaatctatcgaagtgtgtggagaatgtttgcgtggtaggtaaaaacttacaaaagaaacctcggaaaatggacccaaaggcccaaacctcaacgagctagagagagtcacgcggttcgagggagcgtgaggatcaatcacatctacgagaagatattgccatgcagagcaaaaaagcagagacagagagagggaacatgatggaccggtcgcgatgcttcggctgtcgtccaacgaccaaagcaccaatttttacccactttttcctattttcaaatatcttcttcttcttttgctttcaaattattataaatcattatatgatcaacgatcctattataatcattatgctaacacatcggaattcggtctacataacacctttgtctcctaaattcactccatatattctactcggatcaatctacacagaaatcaactatgtaatcactgttaaccagataaaatttgattgtgtaagttgaatccacttgtacaatgacacattgtggtcattattttaagggacaagggttactttacattaagacttcatccaattcgattacgtcgttatctttgaacaaaacaataatatgtttcttcgtcatcttggattcataacaacaaattgttatgagcatccaaagcgagactaaataagtttattaaattgttaaagagttgatcttctatggtatagagatatatcacactgtcaaaaacattttttcgtaaatattggttagttttataatgatacataatacagatgaaactaaatataattcttgtttaaaattctgtacattaagaaatataattatatacatatatgtgaaactagtgataagaaatttaaaccctaactaaagcaagaaaacaaaaagaatcatgtcagtcccaccccatatggaacacaaaccctaataattacatatatgacaattatcttcttataatatatctccatacaaaattattgtatacatatggacatagatatatataaaggttcatccactttaaattttagccatcttcattctcacactcaacccctctctctcctttcattctcattctctctcggctttgttttctctttgatctgattcatctattctatttctatcaccacacaaaaagaagaaaaaaaacaaactttactttaagaattttgaagaataaaatcaaaagagaataacc

SEQ ID NO: 250 - RNA, 21 nt, Artificial sequence
  Synthetic
auaaagguucauccacuuuaa

SEQ ID NO: 251 - RNA, 21 nt, Artificial sequence
  Synthetic
aaaguggaugaaccuuuauau

SEQ ID NO: 252 - RNA, 21 nt, Artificial sequence
  Synthetic
gguucauccacuuuaaauuuu

SEQ ID NO: 253 - RNA, 21 nt, Artificial sequence
  Synthetic
aauuuaaaguggaugaaccuu

SEQ ID NO: 254 - RNA, 21 nt, Artificial sequence
  Synthetic
cuuuaaauuuuagccaucuuc

SEQ ID NO: 255 - RNA, 21 nt, Artificial sequence
  Synthetic
agauggcuaaaauuuaaagug

SEQ ID NO: 256 - RNA, 21 nt, Artificial sequence
  Synthetic
uagccaucuucauucucacac

SEQ ID NO: 257 - RNA, 21 nt, Artificial sequence
  Synthetic
gugagaaugaagauggcuaaa

SEQ ID NO: 258 - RNA, 21 nt, Artificial sequence
  Synthetic
aucuucauucucacacucaac

SEQ ID NO: 259 - RNA, 21 nt, Artificial sequence
  Synthetic
ugagugugagaaugaagaugg

SEQ ID NO: 260 - RNA, 21 nt, Artificial sequence
  Synthetic
ucauucucauucucucucggc

SEQ ID NO: 261 - RNA, 21 nt, Artificial sequence
  Synthetic
cgagagagaaugagaaugaaa

SEQ ID NO: 262 - DNA, 2306 bp, Zea mays
cagggcctagtttgttcggctgatctgtagtcacgtccaggtacattgctgtgattaatggaagacgcgtcactagcaaatacgacaagtgagcgactgtcaactgcgtacatctttttttgtgtggttcactgcttgatgtttctacatgtatatgatgcatggatttgtgatatgctatgtgagtagtcttctttaggaggtgtttagtttttagggactaatttttagtctctccattttattctattttagtccttaaattactaaatacgaaaactacaactctattttagtttctatatttagcaatttagaaactaaaatagaataatatcgagggattaaaaaatagtccatagaaaccaaatacctccttatttaagttgtgaccaaagcgtaagccaggtgtccgtaatgctaaaggtatcgtgacaaatgtccattacctagggaagcaaacaatccgacaacccaagccttcaaatccaagtctttttttttgttatatattatttgaaggttccctgtctcatttttttatctactctaataggtgtcttcaaatcagggcttctagaccctttccactagacatggtgcatgagactcactcactcatcattgagccatatccatccacaattgtttcaaccgaccaagctatatggtgggaagggggggcggacattggcatataagatccaatcgtcacttggccacatccaccatctttccaatacacaaagctatgggggcacatggaggggagctgttgtggggaagtgcactaccggagaacgggacatcagtaccacttaaaaaaaccggttgcagtacaatagaaatcaaatgctctaatctttagtcctggttgcacaaatcgatagtgaaggtaagcctttattgccgggttttggcttgaattggtactaaaagtccaactttagtatcgattcctggcatgaattggtactaaatagggaattttagtaccagtttgagcggtacaaaagtgactttttgttttacatatttataaatgatattgatgaatatttaatatatacacgtactaattaatatatgtacatataaaatgtacaagcaagtagttttttttcaaagggacatgtatataaatattaagggtagctctttccaataggacattaacacaaatatacaagtaaatagtatagtaatatatgtacaataatacatacaaatgaacagcaataatagttcttgaattgcttcaatcttttgcaaagagagtagttctttccttcatctttataaactatgatttcaaaaaaaagtattcttatattaaattcaaagctgaaatactttatcaaacccaaattaaacccaaagagagtagttcgtttctttcttcttcaagtgttcgtcacatcggcattgtacatatatacataaactttccttcctttcttcacgcttcacctttaactcggcactggtgaaaacattatgatagacggaactagcatggattacaacattaataataaatatacgctaaaacattctgatagataaagactgataatgtgatcaatagtacagacaatagttgatatgttgatcgataagagttaggtgcattagagcatctccaataataacaataatacctcaaactggtgtctcaaattgaaatataggactctacacagaaaaactactctaacgatgtcttattttataaaatttgattaaaacagtataaagcattttctcaagtgcctcaaatatattacaccatagtgacttccctataatatagatttatggttttaatgttgaagcagaatgttctttatgccataaattctataaattatatttatttttaaattataaagtatttttatgttatgctgttggagaaccactctcctagtgaacaccaactacccaaagagagtagttctccctctcgcacccacgcggcagcccccacggcgtgctataaatacttcacgaacggcccggatatctccatccctgcatcgcaccctcccgggccgccttctcttctccagcgtccgatctcccactcccctccctcaccgcagctctcccacctccgccctccccccgcacgcgctcgccacctcgccctcccctccacgttgctcgcacccgcgcttatataaggtatgcctcttgccctctccaaacccctccgcgagggcctagggtctggcgtgctgagctggagctgatggatctagggtttgggttgcggtgatggtcctgcagtgcaggaggagctc

SEQ ID NO: 263 - RNA, 21 nt, Artificial sequence
  Synthetic
uuuuauaaaauuugauuaaaa

SEQ ID NO: 264 - RNA, 21 nt, Artificial sequence
  Synthetic
uuaaucaaauuuuauaaaaua

SEQ ID NO: 265 - RNA, 21 nt, Artificial sequence
  Synthetic
uuugauuaaaacaguauaaag

SEQ ID NO: 266 - RNA, 21 nt, Artificial sequence
  Synthetic
uuauacuguuuuaaucaaauu

SEQ ID NO: 267 - RNA, 21 nt, Artificial sequence
  Synthetic
uuaaaacaguauaaagcauuu

SEQ ID NO: 268 - RNA, 21 nt, Artificial sequence
  Synthetic
augcuuuauacuguuuuaauc

SEQ ID NO: 269 - RNA, 21 nt, Artificial sequence
  Synthetic
aauuauaaaguauuuuuaugu

SEQ ID NO: 270 - RNA, 21 nt, Artificial sequence
  Synthetic
auaaaaauacuuuauaauuua